(19) United States
(12) Patent Application Publication
(10) Pub. No.: US 2018/0117574 A1
(43) Pub. Date: May 3, 2018

(54) BIARYL LIGANDS FOR TRANSITION METAL-CATALYZED REACTIONS

(71) Applicant: The Regents of the University of California, Oakland, CA (US)

(72) Inventors: Bruce H. Lipshutz, Santa Barbara, CA (US); Sachin Handa, Louisville, KY (US); Evan Landstrom, Los Angeles, CA (US)

(21) Appl. No.: 15/797,108

(22) Filed: Oct. 30, 2017

Related U.S. Application Data
(60) Provisional application No. 62/414,991, filed on Oct. 31, 2016.

(51) Int. Cl. B01J 31/02; B01J 31/28; C07C 1/32; C07C 11/12

(52) U.S. Cl. CPC B01J 31/0214; B01J 31/28; C07C 2531/28; C07C 11/12; C07C 2531/22; C07C 1/321

(57) ABSTRACT

In one embodiment, the present application discloses ligands of the formula A, wherein the variables are as described herein, and methods for using the ligands in cross-coupling reactions in organic and polar media:

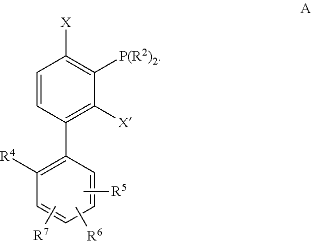

### RELATED APPLICATION

This application claims the priority under 35 USC 119(e) of U.S. Application No. 62/414,991, filed Oct. 31, 2016 which is incorporated into this application by reference.

### STATEMENT REGARDING FEDERALLY SPONSORED RESEARCH

This invention was made with Government support under Grant No. NSF GOALI SusChEM 1566212, awarded by the National Sciense Foundation. The Government has certain rights in this invention.

### BACKGROUND OF THE INVENTION

Transition metal-catalyzed cross-coupling reactions have become one of the most important transformations in organic chemistry. A. de Meijere, F. Diederich, Eds. Metal-Catalyzed Cross-Coupling Reactions, Vol. 2: Wiley-VCH, Weinheim, 2004. J.-P. Corbet, G. Mignani, Chem. Rev. 2006, 106, 2651.

Development of efficient chiral or achiral ligands for metal-catalyzed cross-couplings has gained particular attention in the last twenty years. It has been demonstrated that the ligands play essential roles in the catalytic cycle, including oxidative addition, transmetallation, and reductive elimination. In addition, the steric and electronic properties of the ligand can greatly influence the rate, regioselectivity and stereoselectivity of the cross-coupling reactions. See, for example, S. L. Buchwald et al., J. Am. Chem. Soc. 2005, 127, 4685; S. L. Buchwald et al., Angew. Chem., Int. Ed. 2004, 43, 1871; S. L. Buchwald et al., J. Am. Chem. Soc. 2007, 129, 3358; S. L. Buchwald et al., WO2009/076622; J. F. Hartwig et al., WO 2002/011883; J. F. Hartwig et al., J. Am. Chem. Soc. 1996, 118, 7217; G. C. Fu et al., J. Am. Chem. Soc. 2001, 123, 10099; and Beller et al., Angew. Chem., Int. Ed. 2000, 39, 4153; M. Beller et al., Chem. Comm. 2004, 38. These researchers have developed efficient ligands for cross-coupling reactions forming carbon-carbon, carbon hydrogen, and carbon-heteroatom bond-forming reactions (“cross-coupling reactions”).

The Suzuki-Miyaura coupling reaction is one of most useful methods for the formation of carbon-carbon bonds and has been used in numerous synthetic processes. See N. Miyaura, Topics in Current Chem. 2002, 219, 11 and A. Suzuki, Organomet. Chem. 1999, 576, 147. Despite recent advances on this reaction, Suzuki-Miyaura couplings typically rely on catalyst loadings in the 1-5 mol % (10,000-50,000 ppm) range. Development of new ligands for cross coupling reactions, including Suzuki-Miyaura couplings, that enable both precious metal and non-precious metal catalysts to be used at the ppm level remains an important goal for synthetic chemistry; given the endangered metal status of several common transition metals (e.g., Pd), the need for a reduction in the environmental impact of such processes, the cost of precious metals, and the problems of removal of residual metals in targeted compounds, such as APIs (active pharmaceutical ingredients). Other common cross-couplings to which this invention applies, in particular, include Sonogashira couplings and amination reactions.

Precious metal catalysis in organic synthesis, in large measure, has been and continues to be among the most heavily utilized inroads to C—C, C—H and C-heteroatom bond constructions. Chief among these lies palladium chemistry, and with the 2010 Nobel Prizes recognizing Pd-catalyzed Suzuki, Heck and Negishi couplings, even greater use of these and related processes are to be expected. On the other hand, platinoids, in general, are now regarded as “endangered”; that is, the amount of such metals to which there is economical access is finite, and supplies continue to dwindle. Thus, in a sense, award-winning organopalladium chemistry might be viewed as at odds with sustainability. In one aspect, the coupling reactions utilize organic solvents as the reaction medium, while in another, micelllar catalysis in water is the reaction medium and requires no organic solvents, although organic solvents may be used as “co-solvents.”

### New Approach to Suzuki-Miyaura Couplings

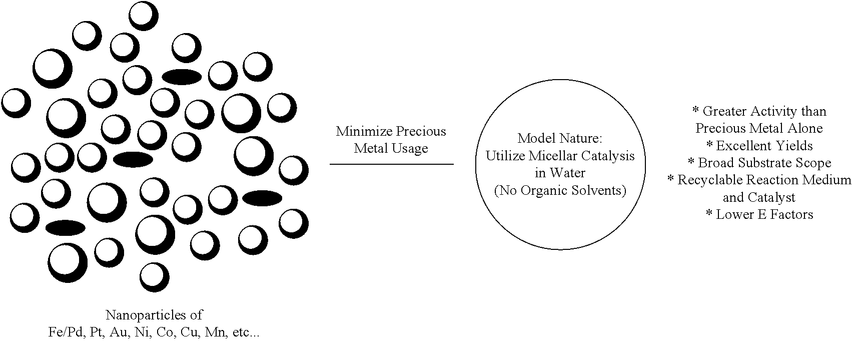

To circumvent this situation, alternative metals have been studied, in particular nickel and copper, especially as applied to the most heavily used Pd-catalyzed cross-coupling: Suzuki-Miyaura reactions. While these have led to varying degrees of success, Pd remains, by far, the metal of choice. Ideally, technology that accomplishes the desired transformations would do so at the ppm level of palladium, in particular at the ≤1,000 ppm level (≤0.1 mol %). Furthermore, utilization of trace levels of this metal, perhaps as found as an “impurity” in other inexpensive metal salts, would ultimately translate into both a recycling of natural sources of Pd while the cost for its use, therefore, approaches zero.

The Buchwald SPhos ligand and the HandaPhos ligand are known to be two commercially available ligands for Pd-catalyzed cross coupling reactions. These ligands, while having been well adopted as highly effective agents for such cross coupling-reactions, are expensive and require several synthetic steps to prepare.

### SUMMARY OF THE INVENTION

Palladacycles have become especially valuable as precursors to important Pd-catalyzed reactions in organic synthesis. Among the many types of reactions their derived ligated Pd(0) forms catalyze, Suzuki-Miyaura cross-couplings are among the most valued. Most, however, are used under traditional conditions that rely on organic solvents as reaction medium. Use of such species in alternative reaction media, such as under aqueous micellar conditions, require each catalyst to be able to gain entry to micelles containing hydrophobic inner cores, and for this, palladacycles that bear additional lipophilic groups on the biaryl framework enhance catalyst activity through enhanced lipophilic interactions. Such phenomena are irrelevant in traditional organic media. By virtue of this switch from organic solvents to nanomicelles that function as nanoreactors, such palladacycle pre-catalysts can now be used at the ppm level, in contrast to typical palladcycles that are ineffective at these low loadings. This not only dramatically reduces the cost for usage of such Pd catalysts, but also extends the available supply of this endangered metal.

Therefore, a continuing need exists for novel and effective ligands, potentially useful at the ppm level of the associated transition metal that are inexpensive and readily available for performing cross-coupling reactions under standard conditions in organic solvents, as well as under the novel conditions utilizing the basic principles of green chemistry (i.e., in water).

The present invention relates to novel monophosphorus ligands prepared from readily available precursors and the preparation of metal complexes comprising these ligands as catalysts for applications to cross-couplings and several related reactions. More particularly, the present invention relates to these phosphine ligands and the catalysts prepared from the phosphine ligands for performing transition metal catalyzed cross-coupling reactions between sp2 and sp3 centers on carbon, as well as sp2 and sp2 centers on carbon, sp and sp2 centers on carbon, and sp and sp3 centers on carbon. These include all the known varieties of carbon-carbon, carbon-hydrogen, and carbon-heteroatom bond forming reactions typically referred to as cross-coupling reactions.

The following embodiments, aspects and variations thereof are exemplary and illustrative but are not intended to be limiting in scope.

One aspect of the present application discloses a technology that takes a readily available, earth-abundant iron salt that contains only parts per million (ppm) levels of Pd, and processes it into a very active catalyst capable of mediating cross-coupling reactions, such as Suzuki-Miyaura cross coupling reactions in the presence of the ligands disclosed herein, that may be performed in organic solvent, in a mixture of water and organic solvent, in water as the predominant reaction medium, or in water as the only reaction medium. In another aspect of the present application is disclosed a technology that utilizes the novel ligands disclosed herein, at the ppm level, for cross coupling reactions in organic solvents.

In one embodiment, there is provided a catalyst composition comprising: a) a reaction solvent or a reaction medium; b) organometallic nanoparticles comprising: i) a nanoparticle (NP) catalyst, prepared by a reduction of an iron salt in an organic solvent, wherein the catalyst comprises at least one other metal selected from the group consisting of Pd, Pt, Au, Ni, Co, Cu, Mn, Rh, Ir, Ru and Os or mixtures thereof; c) a ligand of the formula formula A:

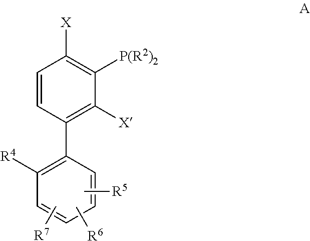

wherein the variables are as defined herein; and d) a transition metal or mixtures of two or more transition metals present in less than or equal to 50,000 ppm relative to the iron salt; or relative to the substrate.

In one variation of the above catalyst composition, the metal or mixtures thereof is present in less than or equal to 40,000 ppm, 30,000 ppm, 20,000 ppm, 10,000 ppm, 5,000 ppm, 3,000 ppm, 2,000 ppm or 1,000 ppm. In another variation, the metal or mixtures thereof is present in less than or equal to 1,000 ppm. In another variation of the composition, the presence of a surfactant provides nanomicelles for housing a substrate. In another variation, the composition may be used in reactions employing standard organic solvents, organic solvents or solvent mixtures and/or organic solvents in polar media or another polar solvent, such as in water. In another variation, the polar solvent or polar reaction medium is water. In yet another variation, the polar solvent or polar reaction medium is a glycol or glycol ether selected from ethyleneglycol, propylene glycol, 2-methoxyethanol, 2-ethoxyethanol, 2-propoxyethanol, 2-isopropoxyethanol, 2-butoxyethanol, 2-phenoxyethanol, 2-benzyloxyethanol, 2-(2-methoxyethoxy)ethanol, 2-(2-ethoxyethoxy)ethanol, 2-(2-butoxyethoxy)ethanol, dimethoxyethane, diethoxyethane and dibutoxyethane; or mixtures thereof. In one variation of the above, the organometallic nanoparticles are present as a complex. In another variation, the reaction medium is a micellar medium or an aqueous micellar medium. In another variation, the catalyst composition further comprises water.

In another embodiment, there is provided an aqueous micellar composition for enabling cross-coupling reactions containing organometallic nanoparticles (NPs) as catalyst, comprising: a) an element selected from the group consisting of Fe, C, H, O, Mg, and a halide, or the entire combination thereof; and b) palladium, or at least one other metal selected from the group consisting of Pt, Au, Ni, Co, Cu and Mn, or a mixture thereof; wherein the catalyst (NPs) is prepared from a reduction of an iron salt in a solvent and in the presence of a ligand using a reducing agent.

In one variation, there is provided an aqueous micellar composition for enabling cross-coupling reactions containing organometallic nanoparticles (NPs) as catalyst, comprising: a) an element selected from the group consisting of Fe, C, H, O, Mg and a halide; and b) palladium, or at least one other metal selected from the group consisting of Pt, Au, Ni, Co, Cu, Mn, Rh, Ir, Ru and Os or a mixture thereof; wherein the catalyst (NPs) is prepared from a reduction of an iron salt in a solvent and in the presence of a ligand as disclosed herein, using a reducing agent, after which the solvent is removed and to which is then added an aqueous solution containing nanomicelles, wherein the palladium is present in less than or equal to 50,000 ppm relative to the iron metal complex, and wherein the ligand is present in an amount, on a mole-to-mole basis, comparable to the levels of iron salt being used. In another embodiment, there is provided a reaction medium comprising an organic solvent, or mixtures of organic solvents, or mixtures of organic solvents containing varying amounts of water, containing a catalyst derived from a transition metal and any of the ligands disclosed herein.

In one embodiment, the application discloses a ligand of the formula A:

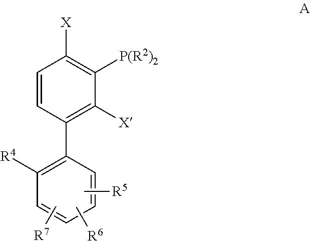

wherein:

X is selected from —OR¹ or —NR′R″ where R′ and R″ is independently selected from the group consisting of H, C₁₋₁₀alkyl, C₃₋₆cycloalkyl, C₆₋₁₄aryl and C₄₋₁₂heteroaryl;

X′ is selected from —OR³ or —NR′R″ where R′ and R″ is independently selected from the group consisting of H, C₁₋₁₀alkyl, C₃₋₆cycloalkyl, C₆₋₁₄aryl and C₄₋₁₂heteroaryl;

each R¹ and R³ is independently selected from a group consisting of C₁₋₁₀alkyl, C₃₋₆cycloalkyl, C₆₋₁₄aryl and C₄₋₁₂heteroaryl;

R² is selected from the group consisting of C₁₋₁₀alkyl, C₃₋₆cycloalkyl, C₆₋₁₄aryl and substituted C₆₋₁₄aryl and C₄₋₁₂heteroaryl;

R⁴ is H or is selected from the group consisting of —OC₁₋₁₀alkyl, C₁₋₁₀alkyl, C₃₋₆cycloalkyl, —SR⁸, —NR⁸R⁹, C₆₋₁₄aryl and C₄₋₁₂heteroaryl;

each R⁵ and R⁶ is H or R⁵ and R⁶ together with the aryl group to which they are attached to form a fused substituted or unsubstituted aromatic ring or heteroaromatic ring;

R⁷ is H or is selected from the group consisting of —OC₁₋₁₀alkyl, C₁₋₁₀alkyl, —SR⁸, —NR⁸R⁹, C₆₋₁₄aryl and C₄₋₁₂heteroaryl; and each R⁸ and R⁹ is independently H or C₁₋₁₀alkyl.

In one variation of the ligand, each R¹ and R³ is independently selected from a group consisting of —CH₃, —CH₂CH₃, CH₂CH₂CH₃, —CH₂CH₂CH₂CH₃, -phenyl, 1-naphthyl and 2-naphthyl. In another variation, R⁴ is a substituted or unsubstituted C₆₋₁₄aryl or a substituted or unsubstituted C₄₋₁₂heteroaryl. In another variation, R⁴ is selected from the group consisting of —OC₁₋₃alkyl, —OC₁₋₆alkyl and C₁₋₃alkyl. In another variation, R⁴ is selected from the group consisting of —OCH₃, —OCH₂CH₃, —CH₃, —CH₂CH₃, —CH₂CH₂CH₃ and —CH₂CH₂CH₂CH₃. In another variation, R⁷ is selected from the group consisting of n-propyl, n-butyl, n-pentyl, n-hexyl, n-heptyl, n-octyl, n-nonyl and n-decyl. In another variation, each R² is independently selected from the group consisting of cyclopentyl, cyclohexyl, t-butyl, substituted or unsubstituted C₆₋₁₄aryl or a substituted or unsubstituted C₄₋₁₂heteroaryl. In one variation of the above, the aryl or heteroaryl ring is substituted by 1 or 2 substituents independently selected from the group consisting of nitro, CF₃—, CF₃O—, CH₃O—, —COOH, —NH₂, —OH, —SH, —NHCH₃, —N(CH₃)₂, —SMe and —CN.

In one aspect, the ligand is of the formula A-1:

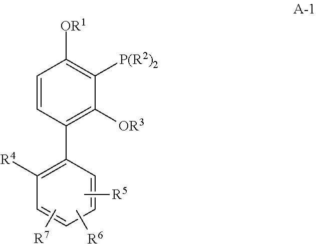

wherein:

each R¹ and R³ is independently selected from a group consisting of C₁₋₁₀alkyl, C₃₋₆cycloalkyl, C₆₋₁₄aryl and C₄₋₁₂heteroaryl;

R² is selected from the group consisting of C₁₋₁₀alkyl, C₃₋₆cycloalkyl, C₆₋₁₄aryl and substituted C₆₋₁₄aryl and C₄₋₁₂heteroaryl

R⁴ is H or is selected from —OC₁₋₁₀alkyl and C₃₋₆cycloalkyl;

each R⁵ and R⁶ is H or R⁵ and R⁶ are each independently an aryl or a heteroaryl ring, or R⁵ and R⁶ together with the aryl group to which they are attached to form a substituted or unsubstituted aromatic ring; and R⁷ is H or is selected from the group consisting of —OC₁₋₁₀alkyl, C₁₋₁₀alkyl, —SR⁸, —NR⁸R⁹, C₆₋₁₄aryl and C₄₋₁₂heteroaryl.

In another aspect of the ligand, R⁵ and R⁶ together form a substituted or unsubstituted aromatic ring or a substituted or unsubstituted heteroaromatic ring. In one variation of the above, the aromatic ring is a phenyl ring or a naphthyl ring, and the heteroaromatic ring is selected from the group consisting of furan, imidazole, oxazole, pyrazine, pyrazole, pyridazine, pyridine and pyrimidine.

In another aspect of the above, the ligand is of the formula B or C:

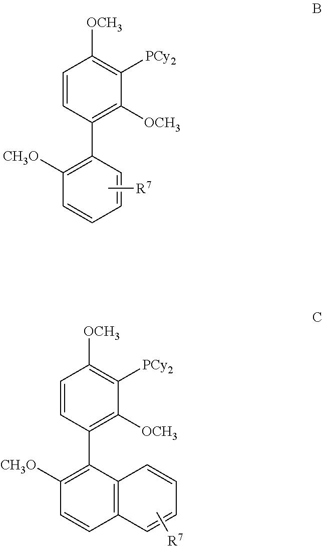

wherein: R⁷ is H or is selected from the group consisting of —OC₁₋₁₀alkyl, C₁₋₁₀alkyl, —SR⁸, —NR⁸R⁹, C₆₋₁₄aryl and C₄₋₁₂heteroaryl.

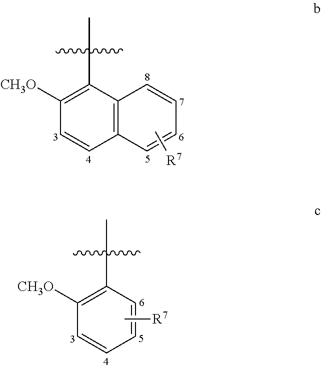

As represented herein, an aryl group such as in b or c showing a substituent position of R⁷ means that for b, R⁷ may be substituted at any of the open position of the phenyl group, such as the 3-phenyl, 4-phenyl, 5-phenyl or 6-phenyl; and for c, R⁷ may be substituted at any of the open position of the phenyl group, such as the 3-naphthyl, 4-napthyl, 5-naphthyl, 6-naphthyl, 7-naphthyl or 8-naphthyl. In certain variations, R⁷ may be substituted in one or independently on both aryl ring of the naphthyl ring.

In another aspect of the compound of formula A, the compound comprises the formulae B-1, B-2 and B-3:

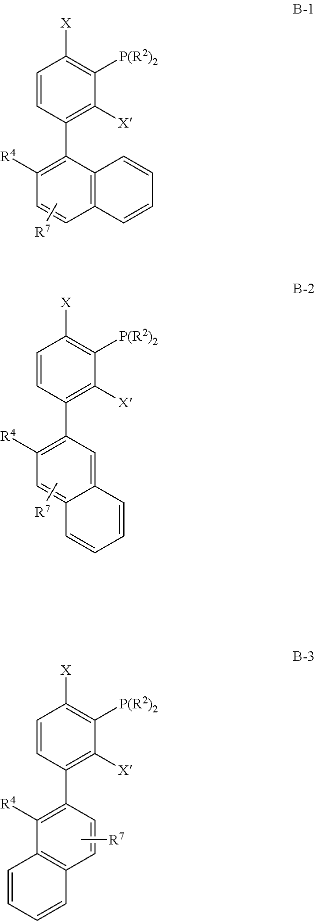

In another aspect, the ligand is selected from the group consisting of A-2 to A-31:

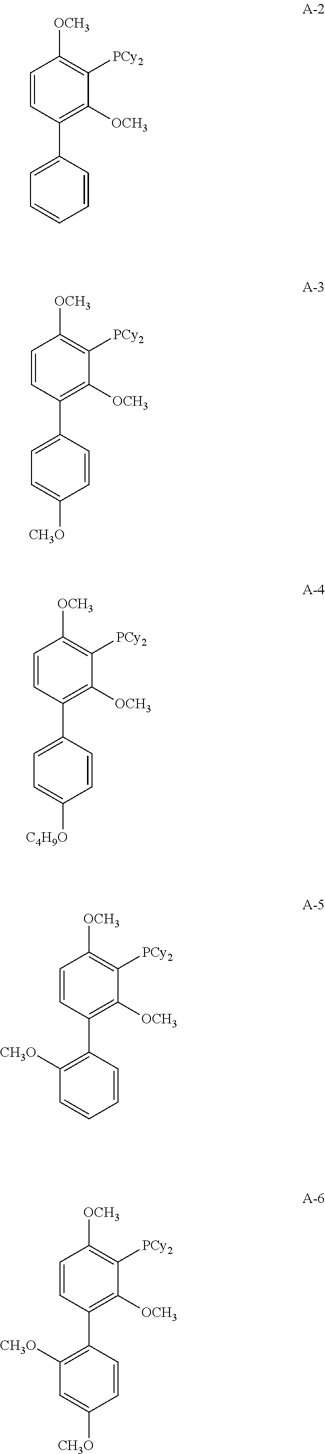

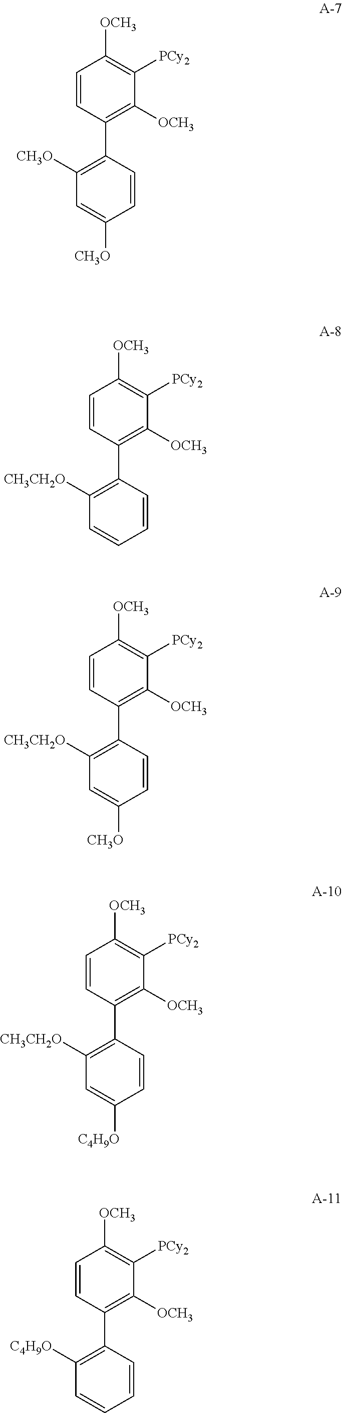

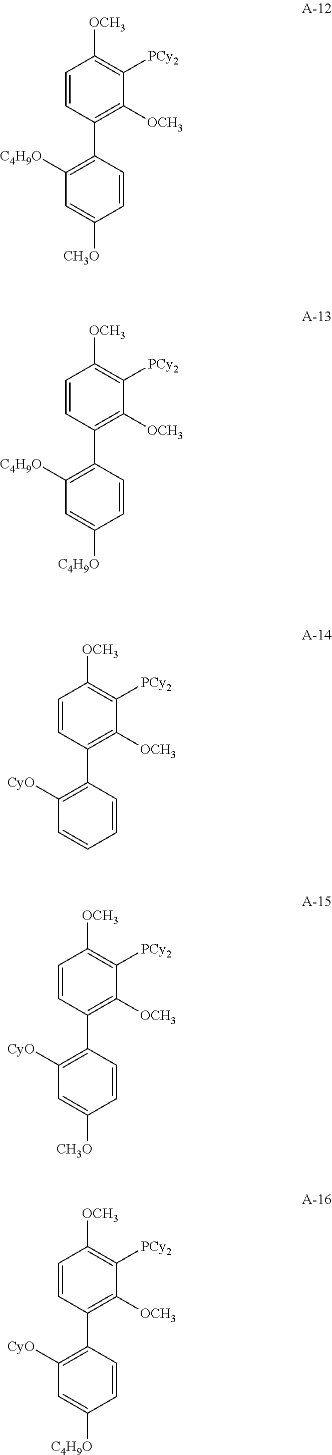

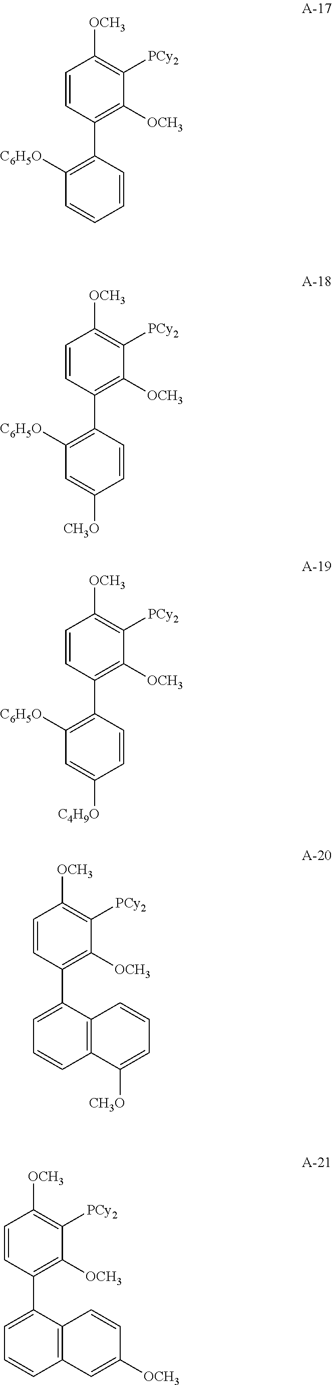

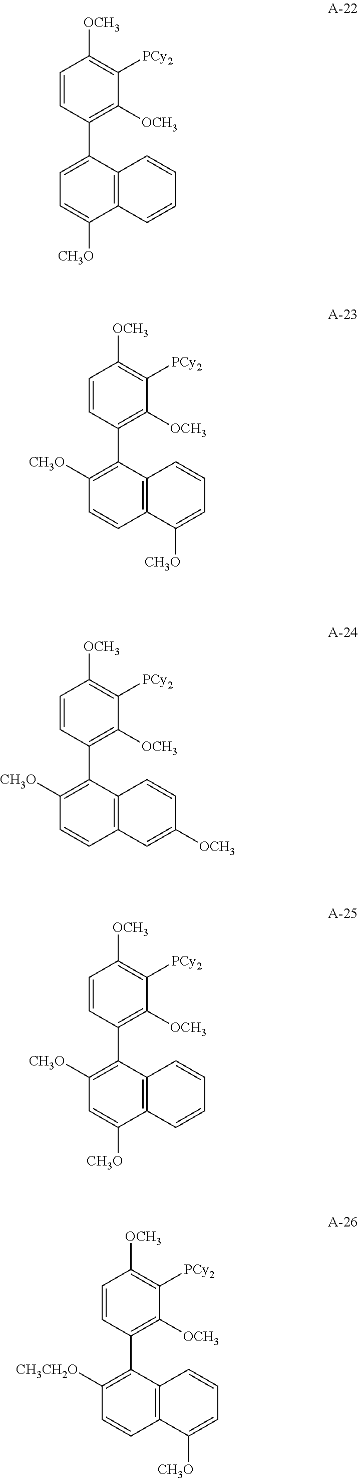

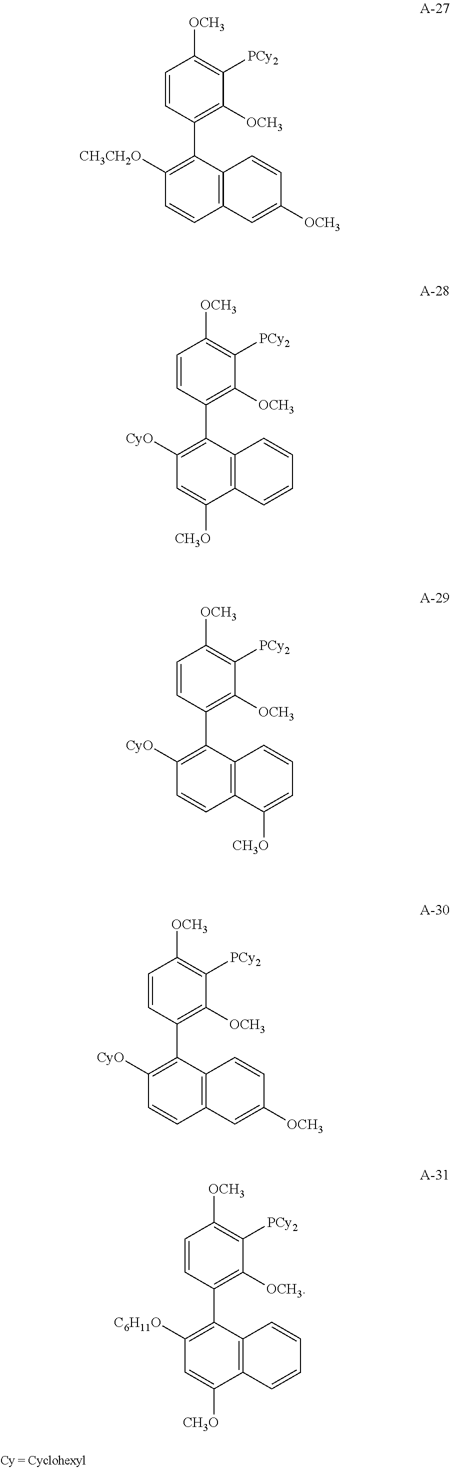

In one embodiment, there is provided a catalyst composition comprising:

a) organometallic nanoparticles comprising:

a nanoparticle (NP) catalyst, prepared by a reduction of an iron salt in an organic solvent, wherein the catalyst comprises at least one other metal selected from the group consisting of Pd, Pt, Au, Ni, Co, Cu, Mn, Rh, Ir, Ru and Os or mixtures thereof;

b) a ligand of the formula A:

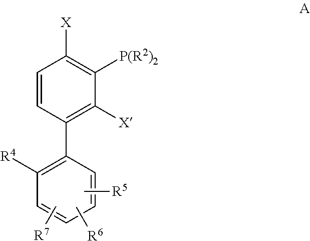

wherein:

X is selected from —OR¹ or —NR′R″ where R′ and R″ is independently selected from the group consisting of H, C₁₋₁₀alkyl, C₃₋₆cycloalkyl, C₆₋₁₄aryl and C₄₋₁₂heteroaryl;

X′ is selected from —OR³ or —NR′R″ where R′ and R″ is independently selected from the group consisting of H, C₁₋₁₀alkyl, C₃₋₆cycloalkyl, C₆₋₁₄aryl and C₄₋₁₂heteroaryl;

each R¹ and R³ is independently selected from a group consisting of C₁₋₁₀alkyl, C₃₋₆cycloalkyl, C₆₋₁₄aryl and C₄₋₁₂heteroaryl;

R² is selected from the group consisting of C₁₋₁₀alkyl, C₃₋₆cycloalkyl and C₆₋₁₄aryl;

R⁴ is H or is selected from the group consisting of —OC₁₋₁₀alkyl, C₁₋₁₀alkyl, —SR⁸, —NR⁸R⁹, C₆₋₁₄aryl and C₄₋₁₂heteroaryl;

each R⁵ and R⁶ is H or R⁵ and R⁶ together with the aryl group to which they are attached to form a substituted or unsubstituted aromatic ring or heteroaromatic ring;

R⁷ is H or is selected from the group consisting of —OC₁₋₁₀alkyl and C₁₋₁₀alkyl, —SR⁸, —NR⁸R⁹, C₆₋₁₄aryl and C₄₋₁₂heteroaryl; and each R⁸ and R⁹ is independently H or C₁₋₁₀alkyl;

c) a surfactant; and

d) a reaction solvent or a reaction medium; wherein the metal or mixtures thereof is present in less than or equal to 50,000 ppm relative to the iron salt.

In another embodiment, there is provided an aqueous micellar composition for enabling cross-coupling reactions containing organometallic nanoparticles (NPs) as catalyst, comprising:

a) an element selected from the group consisting of Fe, C, H, O, Mg, and a halide, or the entire combination thereof; and

b) palladium, or at least one other metal selected from the group consisting of Pt, Au, Ni, Co, Cu and Mn, or a mixture thereof; wherein the catalyst (NPs) is prepared from a reduction of an iron salt in a solvent and in the presence of a ligand using a reducing agent;

wherein the ligand is of the formula A:

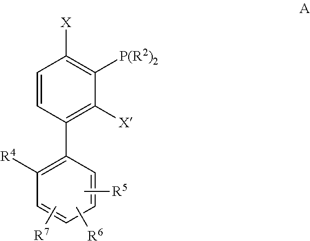

wherein:

X is selected from —OR¹ or —NR′R″ where R′ and R″ is independently selected from the group consisting of H, C₁₋₁₀alkyl, C₃₋₆cycloalkyl, C₆₋₁₄aryl and C₄₋₁₂heteroaryl;

X′ is selected from —OR³ or —NR′R″ where R′ and R″ is independently selected from the group consisting of H, C₁₋₁₀alkyl, C₃₋₆cycloalkyl, C₆₋₁₄aryl and C₄₋₁₂heteroaryl;

each R¹ and R³ is independently selected from a group consisting of C₁₋₁₀alkyl, C₃₋₆cycloalkyl, C₆₋₁₄aryl and C₄₋₁₂heteroaryl;

R² is selected from the group consisting of C₁₋₁₀alkyl, C₃₋₆cycloalkyl, C₆₋₁₄aryl, and substituted C₆₋₁₄aryl and C₄₋₁₂heteroaryl;

R⁴ is H or is selected from the group consisting of —OC₁₋₁₀alkyl, C₁₋₁₀alkyl, C₃₋₆cycloalkyl, —SR⁸, —NR⁸R⁹, C₆₋₁₄aryl and C₄₋₁₂heteroaryl;

each R⁵ and R⁶ is H or R⁵ and R⁶ together with the aryl group to which they are attached to form a substituted or unsubstituted aromatic ring or heteroaromatic ring;

R⁷ is H or is selected from the group consisting of —OC₁₋₁₀alkyl and C₁₋₁₀alkyl, —SR⁸, —NR⁸R⁹, C₆₋₁₄aryl and C₄₋₁₂heteroaryl; and each R⁸ and R⁹ is independently H or C₁₋₁₀alkyl.

In one variation of the catalyst composition, the nanomicelles house, enclose, encase or surround one or more substrates for a catalytic reaction as described herein. In one variation, the ppm, such as 1,000 ppm, is based on a mole to mole basis. In one variation, the relative ppm is determined on a wt/wt basis. In another variation, the other metal is selected from Pd, Pt and Ni, or a mixture thereof.

In one aspect of the above composition, the ligand is of the formula D:

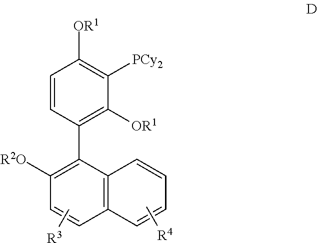

wherein: each R¹ and R² is independently a C₁₋₃alkyl;

R³ is absent or is selected from the group consisting of —OC₁₋₃alkyl, C₁₋₃alkyl and C₃₋₆cycloalkyl; and R⁴ is absent or is selected from the group consisting of —OC₁₋₃alkyl and C₁₋₃alkyl.

In another aspect of the above composition, the iron is selected from the group consisting of a Fe(II) or Fe(III) salt, a Fe(II) salt precursor or Fe(III) salt precursor. In another aspect of the above composition, the palladium is naturally present in the iron salt in amounts less than or equal to 1 ppm, 10 ppm, 50 ppm, 100 ppm, 200 ppm, 300 ppm, 400 ppm or 500 ppm relative to the iron salt or iron complex. As used herein, the term “naturally present” means that the palladium is present in the iron salt as obtained from commercial or natural sources and additional palladium is not added to the iron salt. In another aspect, the amount of Pd present is controlled by external addition of a Pd salt to an iron salt.

A palladacycle of the formula X or Y:

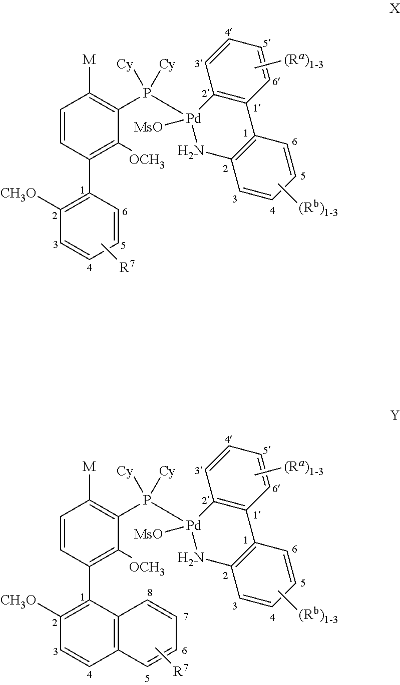

wherein:

for formula X:

M is selected from the group consisting of —OC₁₋₁₀alkyl, —NHC₁₋₁₀alkyl, —N(C₁₋₁₀alkyl)₂ and —NR′R″ where each R′ and R″ is independently H, aryl and heteroaryl;

each R^{a} is independently H or is independently selected from the group consisting of —CF₃, —C₁₋₁₀n-alkyl, -isopropyl, tert-butyl and —OR* where R* is —C₁₋₁₀alkyl or aryl, and may be substituted at the 3′, 4′, 5′ or 6′ position;

each R^{b} is independently H or is independently selected from the group consisting of —CF₃, —C₁₋₁₀n-alkyl, -isopropyl, tert-butyl and —OR* where R* is —C₁₋₁₀alkyl or aryl, and may be substituted at the 3, 4, 5 or 6 position; and

R⁷ is H or is selected from the group consisting of —C₁₋₁₀n-alkyl, -isopropyl, tert-butyl, —OC₁₋₄alkyl, —OCy, —OC₆H₅, and may be substituted at the 3, 4, 5 or 6 position; for formula Y:

M is selected from the group consisting of —OC₁₋₁₀alkyl, —NHC₁₋₁₀alkyl, —N(C₁₋₁₀alkyl)₂ and —NR′R″ where each R′ and R″ is independently H, aryl and heteroaryl;

each R^{a} is independently H or is independently selected from the group consisting of —CF₃, —C₁₋₁₀n-alkyl, -isopropyl and ter-butyl, and may be substituted at the 3′, 4′, 5′ or 6′ position;

each R^{b} is independently H or is independently selected from the group consisting of —CF₃, —C₁₋₁₀n-alkyl, -isopropyl and ter-butyl, and may be substituted at the 3, 4, 5 or 6 position; and R⁷ is H or is selected from the group consisting of —C₁₋₁₀n-alkyl, -isopropyl, tert-butyl, —OC₁₋₄alkyl, —OCy, —OC₆H₅, and may be substituted at the 3, 4, 5, 6, 7 or 8 position.

In one variation of the above formulae and compounds, M is —NR′R″ where each R′ and R″ is independently aryl and heteroaryl. In one variation of the formula X or the formula Y, R^{a} is —C₁₋₁₀n-alkyl, isopropyl or tert-butyl and is substituted in the 4′ position; or is independently —C₁₋₁₀n-alkyl, isopropyl or tert-butyl and is substituted in the 3′ and 5′ positions. In another variation of the formula X, R^{b} is —C₁₋₁₀n-alkyl, isopropyl or tert-butyl and is substituted in the 4′ position; or is independently —C₁₋₁₀n-alkyl, isopropyl or tert-butyl and is substituted in the 3′ and 5′ positions.

In another aspect, there is provided a palladacycle selected from the group consisting of:

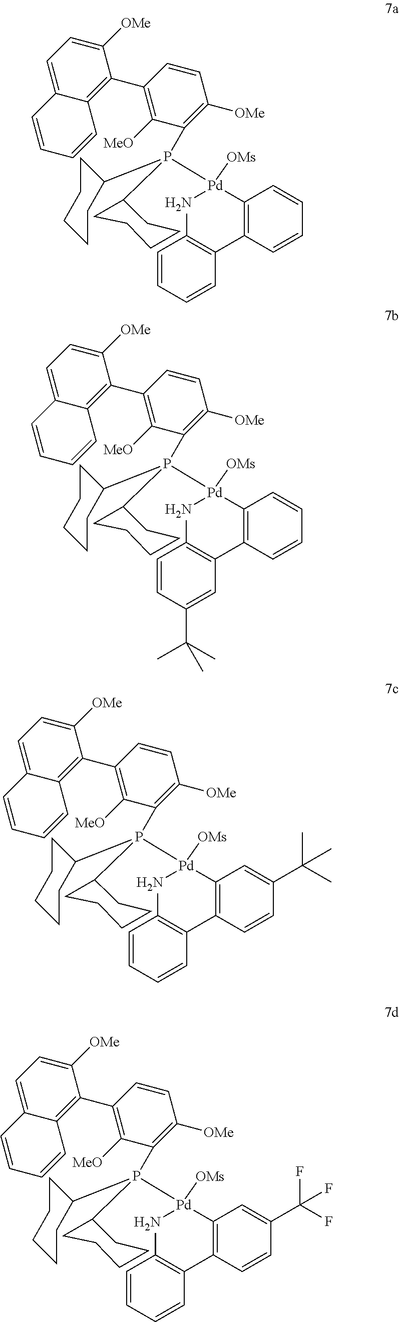

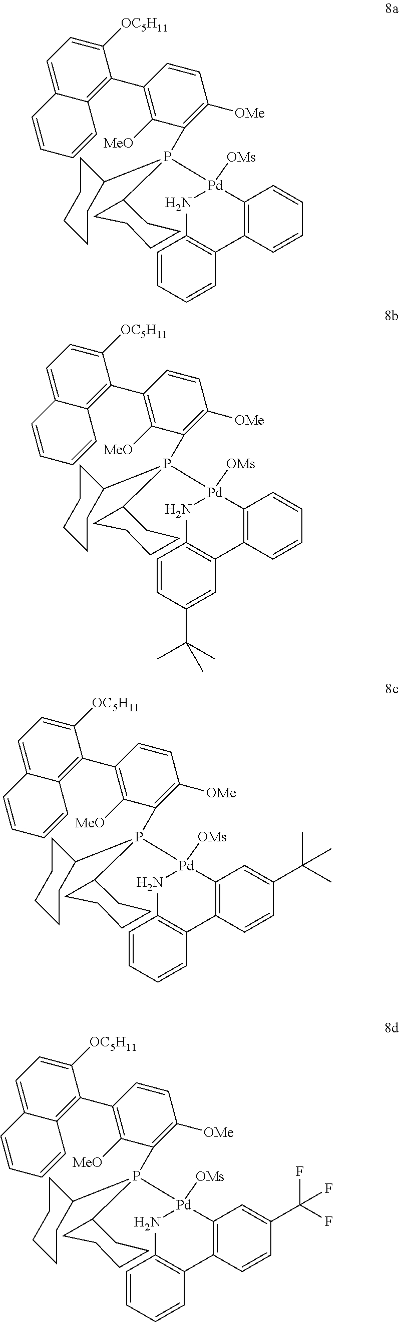

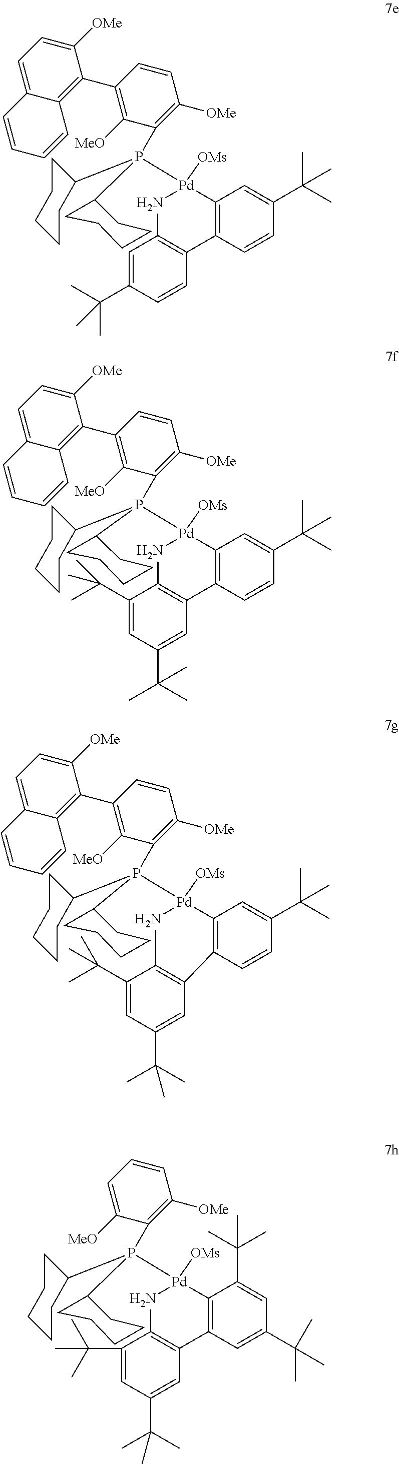

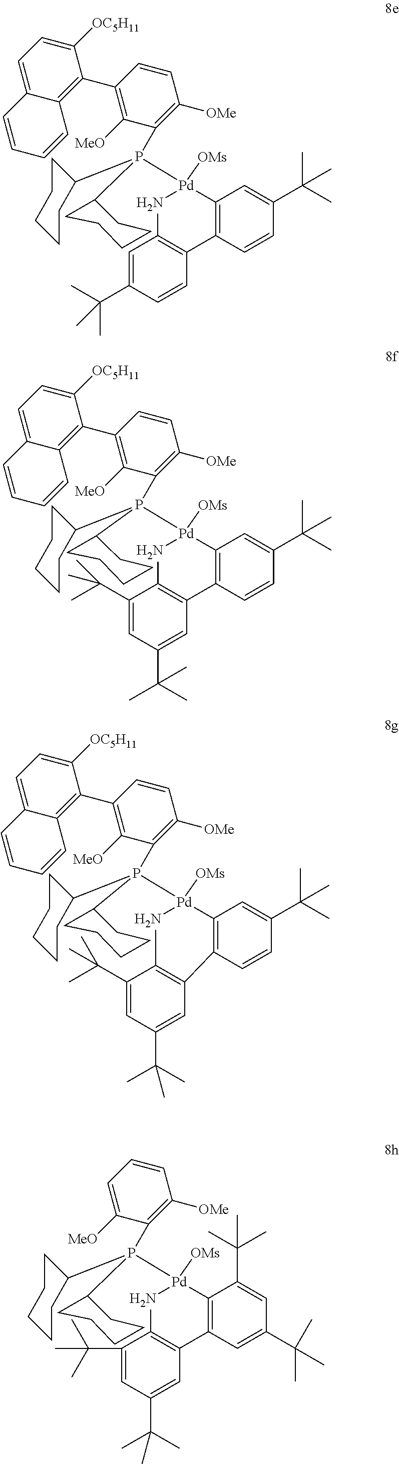

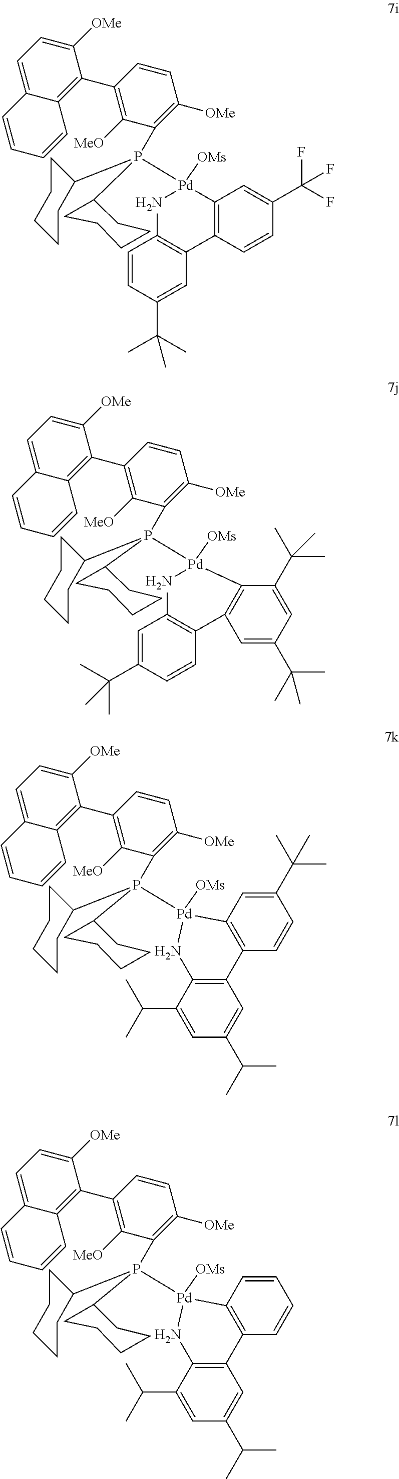

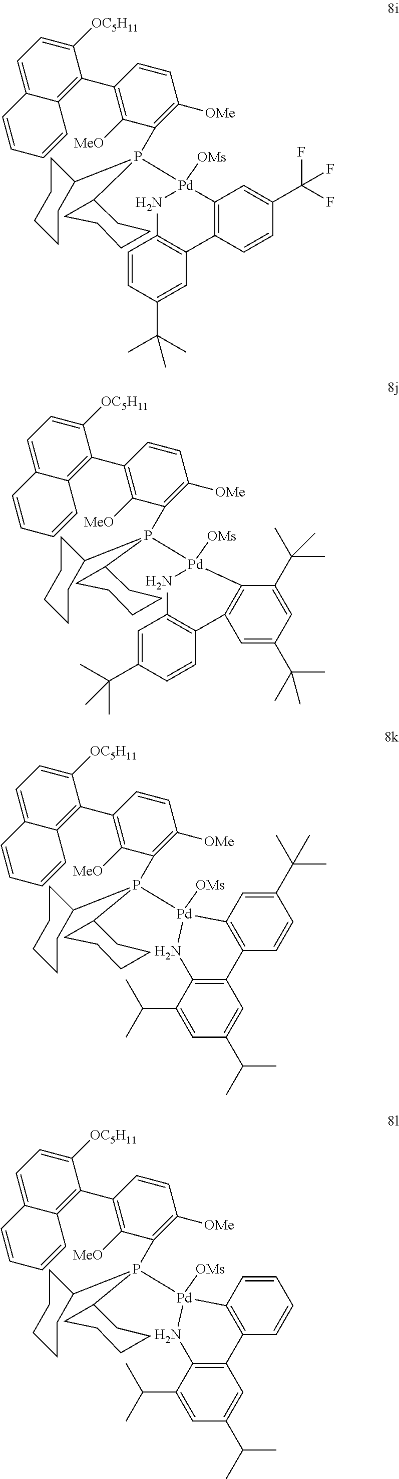

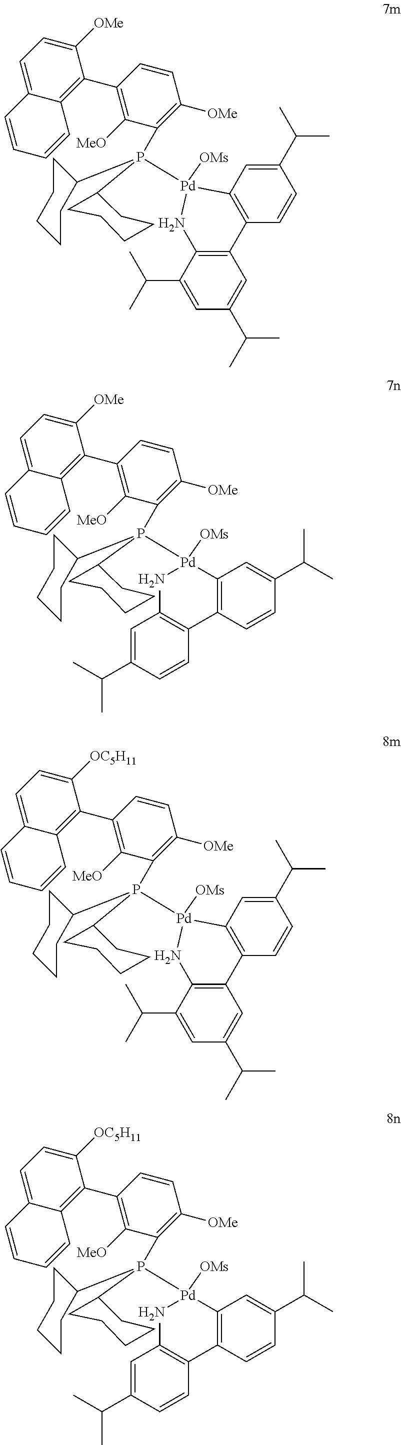

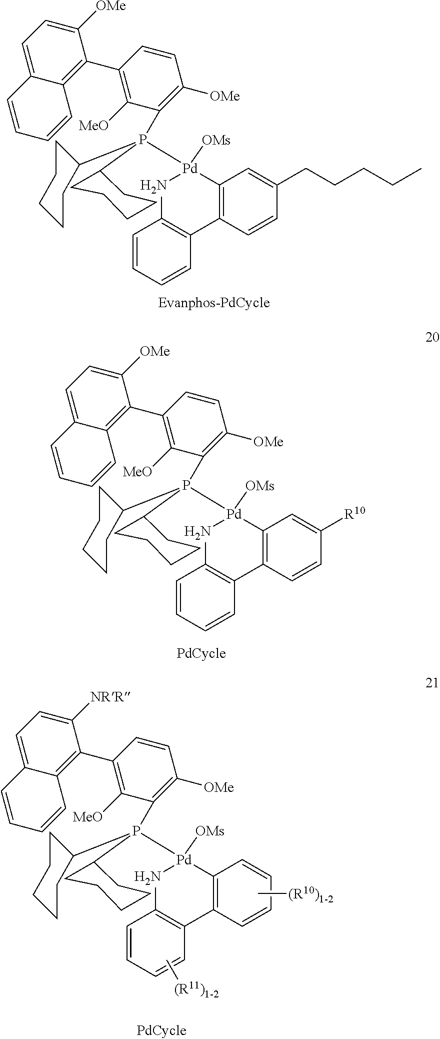

wherein:

each R¹⁰ and R¹¹ is independently H or is selected from the group consisting of —OC₁₋₁₀alkyl, C₁₋₁₀alkyl, —SR⁸, —NR⁸R⁹, C₆₋₁₄aryl and C₄₋₁₂heteroaryl; and

each R′ and R″ is independently H, aryl and heteroaryl.

In another embodiment, there is provided a method for performing a cross coupling reaction between a first coupling substrate of the formula I with a second coupling substrate of the formula II in a reaction condition sufficient to form the coupled product of the formula III:

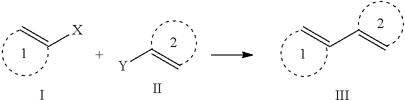

wherein:

X is selected from the group consisting of Cl, Br and I and pseudo halides;

Y is selected from the group consisting of B(OH)₂, B(OR)₂, cyclic boronates, acyclic boronates, B(MIDA), Bpin, BR(OR) and BF₃K, where R is selected from methyl, ethyl, propyl, butyl, isopropyl, ethylene glycol, trimethylene glycol, a cyclic array attaching R to —OR and pinacol;

each of the groups

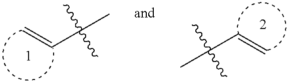

is independently selected from the group consisting of an alkene or a substituted alkene, a cycloalkene or a substituted cycloalkene, an alkyne or a substituted alkyne, an aryl or a substituted aryl, and a heteroaryl or a substituted heteroaryl;

the method comprising:

i) forming a micelle composition comprising aqueous nanoparticles in which the partners I and II are solubilized in water, and an organometallic complex comprising iron nanoparticles, wherein another metal is present in less than 50,000 ppm relative to the limiting substrate of the formula I or formula II, and wherein the composition further comprises a ligand of the formula A:

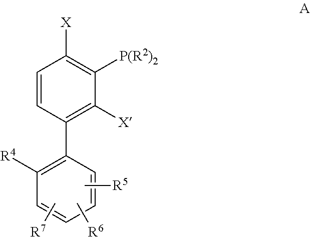

wherein:

X is selected from —OR¹ or —NR′R″ where R′ and R″ is independently selected from the group consisting of H, C₁₋₁₀alkyl, C₃₋₆cycloalkyl, C₆₋₁₄aryl and C₄₋₁₂heteroaryl;

X′ is selected from —OR³ or —NR′R″ where R′ and R″ is independently selected from the group consisting of H, C₁₋₁₀alkyl, C₃₋₆cycloalkyl, C₆₋₁₄aryl and C₄₋₁₂heteroaryl;

each R¹ and R³ is independently selected from a group consisting of C₁₋₁₀alkyl, C₃₋₆cycloalkyl, C₆₋₁₄aryl and C₄₋₁₂heteroaryl;

R² is selected from the group consisting of C₁₋₁₀alkyl, C₃₋₆cycloalkyl, C₆₋₁₄aryl, and substituted C₆₋₁₄aryl and C₄₋₁₂heteroaryl;

R⁴ is H or is selected from the group consisting of —OC₁₋₁₀alkyl, C₁₋₁₀alkyl, C₃₋₆cycloalkyl, —SR⁸, —NR⁸R⁹, C₆₋₁₄aryl and C₄₋₁₂heteroaryl;

each R⁵ and R⁶ is H or R⁵ and R⁶ together with the aryl group to which they are attached to form a substituted or unsubstituted aromatic ring or heteroaromatic ring;

R⁷ is H or is selected from the group consisting of —OC₁₋₁₀alkyl and C₁₋₁₀alkyl, —SR⁸, —NR⁸R⁹, C₆₋₁₄aryl and C₄₋₁₂heteroaryl; and each R⁸ and R⁹ is independently H or C₁₋₁₀alkyl; and ii) contacting the first coupling substrate with the second coupling substrate in water under a condition sufficient to form a product mixture comprising a cross coupling product of the formula III. In one aspect of the method, the metal, other than Pd, is selected from the group consisting of Pt, Au, Ni, Co, Cu, Mn, Rh, Ir, Ru and Os or a mixture thereof.

In one variation of the above composition, the NP catalyst comprising at least one other metal selected from the group consisting of Pd, Pt, Au, Ni, Co, Cu, Mn, Rh, Ir, Ru and Os is naturally present in the iron salt in amounts less than or equal to 1 ppm, 10 ppm, 50 ppm, 100 ppm, 200 ppm, 300 ppm, 400 ppm or 500 ppm relative to the iron complex. In another variation, the iron salt is highly purified iron, such as with an assay as >99.99% trace metal basis, or having less than 0.01% other metals, and the transition metal(s) is (are) added to the composition to be present in the iron salt in an amount that is less than or equal to 1 ppm, 10 ppm, 50 ppm, 100 ppm, 200 ppm, 300 ppm, 400 ppm or 500 ppm relative to or within the iron complex, or the iron salt. In another variation, the transition metal salt is added prior to reduction and NP formation.

In another aspect of the above method, the reaction condition comprises an organic solvent or a mixture of organic solvents or either of these reaction media containing varying percentages of water under a condition sufficient to form a product mixture comprising a cross coupling product of the formula III. In yet another aspect of the method, the reaction condition comprises water and a surfactant, and further comprising an organic solvent as co-solvent. In another aspect of the method, the organic solvent is selected from the group consisting of methanol, ethanol, propanol, isopropanol, butanol(s), n-butanol, 2-butanol, cyclohexane, heptane(s), hexanes, pentanes, isooctane, toluene, xylenes, acetone, amyl acetate, isopropyl acetate, ethyl acetate, methyl acetate, n-butylacetate, methyl formate, diethyl ether, cyclopropyl methyl ether, THF, 2-methyl-THF, acetonitrile, formic acid, acetic acid, ethyleneglycol or PEGs/MPEGs wherein the PEG has a molecular weight range from 300 g/mol to 10,000,000 g/mol, trifluoromethylbenzene, triethylamine, dioxane, sulfolane, MIBK, MEK, MTBE, DMSO, DMF, DMA, NMP and mixtures thereof.

In one variation of the catalyst composition and the method disclosed in the present application, the reaction solvent is water. In another variation, the reaction solvent is a mixture of water and an organic solvent or co-solvent. In one variation, the composition comprises water in an amount of at least 1% wt/wt (weight/weight) of the mixtures. In another embodiment, the water in the mixture is present in an amount of at least 5%, at least 10%, at least 50%, at least 75%, at least 90% or at least 99% wt/wt or more of the mixture. In another variation, the organic co-solvent in the reaction solvent is present in at least 5%, 7%, 10%, 15%, 20%, 30%, 40%, 50%, 70%, 80% or 90% with the remaining being water or a polar solvent. In yet another variation, the organic co-solvent is present at a wt of organic co-solvent to the wt of water (wt/wt) of 1/10, 2/10, 3/10, 5/10, 7/10, 9/10, 10/10, 12/10, 15/10, 17/10, 20/10, 25/10, 30/10, 35/10, 50/10, 60/10, 70/10, 80/10, 90/10, 100/10, 150/10, 200/10, 250/10, 300/10, 400/10, 500/10, 600/10, 700/10, 800/10, 900/10, 1,000/10, 5,000/10 and 10,000/10. In one variation, the reaction may be performed in one of the above reaction solvent composition by wt/wt (e.g., 1/10, organic solvent to water), as a first solvent composition, and when the reaction is completed, the reaction solvent composition may be changed to another composition or second wt/wt composition (e.g., 150/10), to facilitate at least one of the processing of the reaction mixture; transferring of reaction mixture, isolating components of the reaction mixture including the product, minimizing the formation of emulsions or oiling out of the reactants and/or products, and providing an increase in the reaction yields; or a combination thereof. Depending on the reaction or processing steps, the reaction mixture may be changed to a third or other, subsequent solvent composition. In another aspect, water is the only reaction medium in the mixture. In another aspect, non-exclusive examples of the organic solvent or co-solvent may include C₁-C₆ alcohols such as methanol, ethanol, propanol, isopropanol, butanol(s), n-butanol, 2-butanol, etc. . . . , hydrocarbons such as cyclohexane, heptane(s), hexanes, pentanes, isooctane, and toluene or xylenes, or acetone, amyl acetate, isopropyl acetate, ethyl acetate, n-butyl acetate, methyl acetate, methyl formate, diethyl ether, cyclopropyl methyl ether, THF, 2-methyl-THF, acetonitrile, formic acid, acetic acid, ethyleneglycol or PEGs/MPEGs of any length of ethylenoxy units, trifluoromethylbenzene, triethylamine, dioxane, sulfolane, MIBK, MEK, MTBE, DMSO, DMF, DMA, NMP or mixtures thereof.

In one variation of the above composition or the above method, the surfactant is selected from the group consisting of TPGS-500, TPGS-500-M, TPGS-750, TPGS-750-M, TPGS-1000 and TPGS-1000-M, Nok and PTS. In one variation of the above, the surfactant is selected from the group consisting of Triton X-100, Poloxamer 188, Polysorbate 80, Polysorbate 20, Solutol HS 15, PEG-40 Hydrogenated castor oil (Cremophor RH40), PEG-35 Castor oil (Cremophor EL), PEG-8-glyceryl capylate/caprate (Labrasol), PEG-32-glyceryl laurate (Gelucire 44/14), PEG-32-glyceryl palmito stearate (Gelucire 50/13); Polysorbate 85, Polyglyceryl-6-dioleate (Caprol MPGO), Sorbitan monooleate (Span 80), Capmul MCM, Maisine 35-1, Glyceryl monooleate, Glyceryl monolinoleate, PEG-6-glyceryl oleate (Labrafil M 1944 CS), PEG-6-glyceryl linoleate (Labrafil M 2125 CS), Propylene glycol monocaprylate (e.g. Capmul PG-8 or Capryol 90), Propylene glycol monolaurate (e.g., Capmul PG-12 or Lauroglycol 90), Polyglyceryl-3 dioleate (Plurol Oleique CC497), Polyglyceryl-3 diisostearate (Plurol Diisostearique) and Lecithin, and mixtures thereof. Likewise, ionic surfactants, such as SDS (anionic) or CTAB (cationic) surfactants, as well as zwitterionic surfactants can also be used as micellar-forming species in water.

In one variation of the above, there is provided a composition, such as the catalyst composition, prepared by the above described process. In one variation of the above, the halide is Cl or Br. In another variation, the other metal may be present in any of their oxidation states, including 1, 2, 3, 4 or 5. In one aspect of the above composition or method, the reducing agent is a Grignard reagent. In one variation of the above, the reduction of the iron salt is performed in an ether solvent. In another variation, the ether solvent is selected from the group consisting of methyl ether, ethyl ether, THF, Me-THF, dioxane, mono-glyme and di-glyme. In one variation of the above, the solvent is selected from the group consisting of THF, Methyl-THF, toluene, i-PrOAc, MTBE and mixtures thereof. In another variation, the reduction is performed at a temperature between −25° C. and 25° C. In one aspect, the ligand is present in about a 1:1; 1:1.1; 1:1.2; 1:1.3; 1:1.4 or 1:1.5; or 1.5:1; 1.4:1; 1.3:1; 1.2:1; 1.1:1 on a mole-to-mole basis to the levels of iron salt being used. In one embodiment, there is provided a composition prepared by the above method.

In another aspect of the above composition or the above method, the iron is selected from the group consisting of a Fe(II) or Fe(III) salt as precursor to the catalyst. In another aspect of the composition, the palladium is naturally present in the iron salt in amounts less than or equal to 500 ppm relative to the iron complex.

In one variation of the composition or method, the Pt, Au, Ni, Co, Cu, Mn, Rh, Ir, Ru and Os or a mixture thereof is naturally present in less than or equal to 500 ppm (0.05 mole %) within the iron salt. In another variation of the above, the Pt, Au, Ni, Co, Cu, Mn, Rh, Ir, Ru and Os or a mixture thereof is added to the composition in less than or equal to 1,000 or 500 ppm within the iron salt.

In another aspect, the amount of Pd present is controlled by external addition of a Pd salt to an iron salt prior to reduction and NP formation. In one variation of the composition or the method, the iron is a purified iron salt, such as a highly purified iron salt, such as FeCl₃ or a highly purified FeCl₃. In another variation, the iron salt, such as FeCl₃, has an assay of >99.99% trace metal basis, or less than 0.01% other metals. In another variation, the iron salt is purified with substantially no palladium. In one variation of the above, the amount of Pd present by external addition of a Pd salt may be about 1-50,000 ppm, 1-1,000 ppm, 1-500 ppm, 1-300 ppm or 1-200 ppm; 100 ppm, 200 ppm, 300 ppm, 500 ppm or 1000 ppm or more.

In another aspect of the composition, the reducing reagent is a Grignard reagent selected from the group consisting of MeMgCl, MeMgBr, MeMgI, EtMgCl, EtMgBr, EtMgI, i-PrMgCl, i-PrMgBr, i-PrMgI, PhMgCl, PhMgBr, PhMgI, n-hexyl-MgBr, n-hexyl-MgCl, n-hexyl-MgBr, n-hexyl-MgCl, n-hexyl-MgI, NaBH₄, liBH₄, BH₃-THF, BH₃—SMe₂, borane, DIBAL-H and LiAlH₄; and mixtures thereof. In one variation of the composition, the Grignard reagent is in an ethereal solvent. In another variation, the solvent is THF.

In another aspect, the surfactant is selected from the group consisting of TPGS-500, TPGS-500-M, TPGS-750, TPGS-750-M, TPGS-1000 and TPGS-1000-M, Nok and PTS. In one variation of the composition, the surfactant is selected from the group consisting of Triton X-100, Poloxamer 188, Polysorbate 80, Polysorbate 20, Solutol HS 15, PEG-40 Hydrogenated castor oil (Cremophor RH40), PEG-35 Castor oil (Cremophor EL), PEG-8-glyceryl capylate/caprate (Labrasol), PEG-32-glyceryl laurate (Gelucire 44/14), PEG-32-glyceryl palmitostearate (Gelucire 50/13); Polysorbate 85, Polyglyceryl-6-dioleate (Caprol MPGO), Sorbitan monooleate (Span 80), Capmul MCM, Maisine 35-1, Glyceryl monooleate, Glyceryl monolinoleate, PEG-6-glyceryl oleate (Labrafil M 1944 CS), PEG-6-glyceryl linoleate (Labrafil M 2125 CS), Propylene glycol monocaprylate (e.g. Capmul PG-8 or Capryol 90), Propylene glycol monolaurate (e.g., Capmul PG-12 or Lauroglycol 90), Polyglyceryl-3 dioleate (PlurolOleique CC497), Polyglyceryl-3 diisostearate (Plurol Diisostearique) and Lecithin, or mixtures thereof. In another variation, the surfactant is selected from the group consisting of Solutol HS 15, PEG-40 Hydrogenated castor oil (Cremophor RH40), PEG-35 Castor oil (Cremophor EL), Polysorbate 85, or mixtures thereof. In another variation, the surfactant is present as a 0.1 to 20 weight % in water, 1 to 5 weight % in water, or 2 weight % in water. In another aspect, the composition further comprises an organic solvent.

In another aspect of the composition, the iron metal complex as nanoparticles is heterogeneous and can be isolated from the composition, stored and recycled and re-use. In one variation of the method, the metal or mixtures thereof is present in less than or equal to 40,000 ppm, 30,000 ppm, 20,000 ppm, 10,000 ppm, 5,000 ppm, 3,000 ppm, 2,000 ppm or 1,000 ppm.

In one variation of the above method, the other metal (i.e., the “another metal” that is other than iron cited above) is palladium. In another variation, the other metal is present in less than 700 ppm, 500 ppm or 300 ppm. In one variation of the method, the micelle composition is a catalyst composition comprising an aqueous micellar medium together with organometallic nanoparticles as a complex, comprising: a) a surfactant, providing nanomicelles for housing a substrate; b) a nanoparticle (NP) catalyst, prepared by a reduction of an iron salt, wherein the catalyst comprises at least one other metal selected from the group consisting of Pd, Pt, Au, Ni, Co, Cu, Mn, Rh, Ir, Ru and Os or a mixture thereof; c) a ligand; and d) water; wherein the metal or a mixture thereof is present in less than or equal to 50,000 ppm or 1,000 ppm relative to the substrate. In another variation of the method, the coupling reaction is performed between room temperature, or about 20° C. and 50° C.

In another aspect of the above method, the metal, other than Pd, is selected from the group consisting of Pt, Au, Ni, Co, Cu, Mn, Rh, Ir, Ru and Os or a mixture thereof. In another aspect, the method further comprises: iii) contacting the product mixture with an organic solvent to form an organic phase and an aqueous phase; and iv) separating the organic phase from the aqueous phase containing the micelle composition as well as the iron/ppm Pd nanoparticles.

In another aspect, the method further comprising: v) re-cycling the aqueous phase containing the micelle composition and Fe/ppm Pd nanoparticles for use in a subsequent cross coupling reaction. In another aspect of the method, the source of iron is selected from FeCl₃, impure FeCl₃ and mixtures thereof, and the reducing agent is a Grignard reagent. Impure FeCl₃ include 96%, 97%, 98%, 99%, 99% or >99% purity. In another aspect of the method, the reaction condition is a Suzuki-Miyaura coupling condition or a Sonogashira coupling condition, or other common Pd-catalyzed cross-coupling reactions. In one variation of the above, the reaction is an amination reaction, Stille couplings, Negishi couplings, Hiyama couplings and cross-couplings involving oxygen nucleophiles. In another aspect of the method, the reaction is performed at room temperature. In one variation of the above, the reaction is performed at about 20 to 65° C., 20 to 45° C., or 15 to 35° C.

In yet another aspect of each of the above, the method further comprises removal of the solvent in vacuo, and further isolating the nanoparticles from the reaction mixture for re-use or recycling. In another aspect, the method further comprises removal of the solvent in vacuo, and further isolating the nanoparticles from the reaction mixture for re-use or recycling. In one variation, the nanoparticles may be re-use or recycled for 2, 3, 4, 5 or more reactions or processes.

In one variation of the above, the nanoparticles (NPs) are powders generated and used in situ, or isolated and storable at room temperature under an inert atmosphere. In another variation of the composition, the Pd is present in any of its oxidation states, such as Pd^{o}, Pd(I), Pd(II) or Pd(IV). In one variation, the metal is a trace impurity (e.g., in ppm) in the iron salt. In another variation, the metal is added to the composition comprising the organometallic complex before the addition of the reducing agent.

In one embodiment, the application discloses composites or compositions comprising nanoparticles (NPs) as isolable powders derived from an iron (Fe) metal, such as an Fe(II) salt or an Fe(III) salt. In one aspect, the NPs contain primarily C, H, O, Mg, halogen and Fe in their matrix. In another aspect, these NPs may also contain ppm levels of other metals, especially transition metals (e.g., Pd, Pt, Au, Ni, Co, Cu, Mn, Rh, Ir, Ru and Os, and mixtures thereof), that may be either present in the Fe(II) or Fe(III) salts or the transition metals may be added externally prior to reduction (e.g., using Pd(OAc)₂, etc.). In one variation, the transition metal is Pd, Pt or Ni, or a mixture thereof. In the resulting composite, these NPs may be used as heterogeneous catalysts, in an aqueous micellar medium. In another aspect, the NPs maybe used to mediate transition metal-catalyzed reactions. Such metal-catalyzed reactions may include reactions that are catalyzed by Pd (e.g., Suzuki-Miyaura and Sonogashira couplings, etc.), as well as reductions of selected functional groups (e.g., aryl/heteroaryl nitro groups).

In one variation of the above composition, the nanoparticle organometallic complex consists mainly of iron. In another variation, the nanoparticle organometallic complex comprises of a mixture of metals wherein at least 90% wt/wt, 95% wt/wt, 97% wt/wt, 98% wt/wt, 99% wt/wt, 99.5% wt/wt, 99.8% wt/wt or 99.9% wt/wt of the metal is iron. In one variation of the complex, the other metal present in the mixture is palladium.

In one aspect of the composition, the iron metal is selected from the group consisting of a Fe(II) or Fe(III) salt or salt precursor. In one variation, the iron metal is FeCl₃. In another aspect, the palladium is present in the iron metal complex in amounts less than or equal to 400 ppm relative to the iron metal complex. In one variation, the palladium is Pd⁰, prepared by reduction of Pd(OAc)₂ or other Pd salts. In another variation, the iron metal complex is doped by addition of the palladium, before or after reduction of the iron salt. In another variation of the above composition, the palladium is present in less than about 800 ppm, less than 700 ppm, less than 600 ppm, less than 500 ppm, less than 400 ppm, less than 300 ppm, less than 200 ppm or less than 100 ppm.

In another variation, the iron metal complex as nanoparticles is heterogeneous and can be isolated, stored and recycled. In one variation of the above composition, the nanoparticle complex may be stored at room temperature for at least 1 month, 2 month, 3 months, 4 months, 5 months or more than 6 months without any noticeable degradation.

In one variation, the compound of the formula I and the compound of the formula II may also include any sp2-sp2 combination, including cyclopropyl arrays. In one aspect of the above, the metal is Pd, Pt or Ni, or mixtures thereof. In another variation of the above, the iron metal complex containing palladium metal as nanoparticles is present at less than about 10 mol percent (mol %), 8 mol percent, 6 mol percent, 5 mol percent, 3 mol percent, 2 mol percent or less than about 1 mole percent relative to the first coupling substrate of the formula I or the second coupling substrate of the formula II.

In another variation of the above, the substituent is 1, 2 or 3 substituents selected from the group consisting of —OCH₃, —CF₃, —NR¹R², —CH(OC₁₋₆ alkyl)₂, —C(O)NR¹R², —CHO, —CO₂C₁₋₁₂ alkyl, —CO₂C₆₋₁₂ aryl, —CO₂C₃₋₁₀heteroaryl, —C(O)C₆₋₁₂ aryl, —C(O)C₃₋₁₀heteroaryl, wherein each R¹ and R² is independently selected from H and C₁₋₆ alkyl.

In another aspect of the above, the method further comprises: iii) contacting the product mixture with an organic solvent to form an organic phase and an aqueous phase; iv) separating the organic phase from the aqueous phase containing the micelle composition as well as the iron/ppm Pd nanoparticles.

In another aspect of the above, the method further comprises v) re-cycling the aqueous phase containing the micelle composition and Fe/ppm Pd nanoparticles for use in a subsequent cross coupling or other reactions. In another aspect of the above method, the source of iron is selected from FeCl₃, impure FeCl₃ and mixtures thereof. In another aspect, the reducing agent is a Grignard reagent. In one variation of the above, there is provided a catalyst composition prepared by the above described process.

In one variation, the method relates to the choice and source of the iron salt, the method for its conversion to nanoparticles, and the use of micellar catalysis conditions. In one aspect, the treatment of FeCl₃ or impure FeCl₃ with an equivalent of MeMgCl in a minimal amount of a solvent, such as an ether, such as THF, at room temperature affords nanoparticles that, after solvent removal in vacuo, can be used directly in a cross coupling reaction. Alternatively, these particles may be isolated, such as by filtration, and stored at room temperature for months without any noticeable degradation.

In addition to the exemplary embodiments, aspects and variations described above, further embodiments, aspects and variations will become apparent by reference to the drawings and figures and by examination of the following descriptions. The disclosed examples of the related art and limitations are intended to be illustrative and not exclusive. Other limitations of the related art will become apparent to those of skill in the art upon a reading of the specification and a study of the drawings or figures as provided herein.

### DETAILED DESCRIPTION OF THE INVENTION

### Definitions

Unless specifically noted otherwise herein, the definitions of the terms used are standard definitions used in the art of organic synthesis. Exemplary embodiments, aspects and variations are illustratived in the figures and drawings, and it is intended that the embodiments, aspects and variations, and the figures and drawings disclosed herein are to be considered illustrative and not limiting.

An “alkyl” group is a straight, branched, saturated or unsaturated, aliphatic group having a chain of carbon atoms, optionally with oxygen, nitrogen or sulfur atoms inserted between the carbon atoms in the chain or as indicated. A (C₁-C₂₀)alkyl, for example, includes alkyl groups that have a chain of between 1 and 20 carbon atoms, and include, for example, the groups methyl, ethyl, propyl, isopropyl, vinyl, allyl, 1-propenyl, isopropenyl, ethynyl, 1-propynyl, 2-propynyl, 1,3-butadienyl, penta-1,3-dienyl, penta-1,4-dienyl, hexa-1,3-dienyl and hexa-1,3,5-trienyl. An alkyl group may also be represented, for example, as a —(CR¹R²)ₘ— group where R¹ and R² are independently hydrogen or are independently absent, and for example, m is 1 to 8 or 1 to 10, and such representation is also intended to cover both saturated and unsaturated alkyl groups.

An alkyl as noted with another group such as an aryl group, represented as “arylalkyl” for example, is intended to be a straight, branched, saturated or unsaturated aliphatic divalent group with the number of atoms indicated in the alkyl group (as in (C₁-C₂₀)alkyl, for example) and/or aryl group (as in (C₅-C₁₄)aryl, for example) or when no atoms are indicated means a bond between the aryl and the alkyl group. Nonexclusive examples of such group include benzyl and phenethyl.

An “aryl” is a monocyclic or polycyclic ring assembly where each ring is aromatic or when fused with one or more rings forms an aromatic ring assembly. Examples of aryl rings include phenyl, naphthlyl, anthracenyl and phenanthrenyl. If one or more ring atoms is not carbon such as an N or S, then the aryl is a heteroaryl. C_{X} aryl and C_{X-Y} aryl are used where X and Y indicate the number of atoms in the ring.

An “aromatic” is a group where the constituent atoms make up an unsaturated ring system, all atoms in the ring system are sp² hybridized and the total number of pi electrons is equal to 4n+2. An aromatic ring may be such that the ring atoms are only carbon atoms or may include carbon and non-carbon atoms, such as a heteroaryl, including pyridine, thiophene, furan, carbazole, indole, isoindole and as defined herein.

A “bicycloalkyl” means a saturated or partially unsaturated fused bicyclic or bridged polycyclic ring assembly.

A “bicycloaryl” means a bicyclic ring assembly where the rings are linked by a single bond or are fused and at least one of the rings of the assembly is aromatic. A C_{X}bicycloaryl and C_{X-Y}bicycloaryl are used where X and Y indicate the number of carbon atoms in the bicyclic ring assembly and directly attached to the ring. Examples of a bicycloaryl ring include, for example, naphthyl and anthracenyl. A bicycloaryl ring can also be a heterobicyclyl ring where the ring assembly may contain one or more heteroatom such as N or S, such as benzo[b]furan, benzo[b]thiophene, benzimidazole, quinazoline, indolizine, quinoline, isoquinoline, phthalazine, quinoxaline, naphthyridine, quinolizine, indole, isoindole, indazole, indoline, benzoxazole, benzopyrazole, benzothiazole, pteridine, purine, acridine, phenazine and phenoxazine.

An “alkylene” group is a straight, branched, saturated or unsaturated aliphatic divalent group with the number of atoms indicated in the alkyl group; for example, a —(C₁₋C₃)alkylene- or —(C₁₋C₃)alkylenyl-.

A “cyclyl” such as a monocyclyl or polycyclyl group includes monocyclic, or linearly fused, angularly fused or bridged polycycloalkyl or combinations thereof. Such cyclyl group is intended to include the heterocyclyl analogs. A cyclyl group may be saturated, partically saturated or aromatic.

“Halogen” or “halo” means fluorine, chlorine, bromine or iodine.

A “heterocyclyl” or “heterocycle” is a cycloalkyl wherein one or more of the atoms forming the ring is a heteroatom that is a N, O, or S. Non-exclusive examples of heterocyclyl include piperidyl, 4-morpholyl, 4-piperazinyl, pyrrolidinyl, 1,4-diazaperhydroepinyl and 1,3-dioxanyl.

“Substituted or unsubstituted” or “optionally substituted” means that a group such as, for example, alkyl, aryl, heterocyclyl, (C₁-C₈)cycloalkyl, hetrocyclyl(C₁-C₈)alkyl, aryl(C₁-C₈)alkyl, heteroaryl, heteroaryl(C₁-C₈)alkyl, and the like, unless specifically noted otherwise, may be unsubstituted or, may substituted by 1, 2 or 3 substitutents selected from the group such as halo, —NO₂, CF₃—, CF₃O—, CH₃O—, —COOH, —NH₂, —OH, —SH, —NHCH₃, —N(CH₃)₂, —SMe and cyano.

### EXPERIMENTAL

### Synthesis of the Ligands:

The following procedures may be employed for the preparation of the compounds of the present invention. The starting materials and reagents used in preparing these compounds are either available from commercial suppliers such as the Aldrich Chemical Company (Milwaukee, Wis.), Bachem (Torrance, Calif.), Sigma (St. Louis, Mo.), or are prepared by methods well known to a person of ordinary skill in the art, following procedures described in such references as Fieser and Fieser's Reagents for Organic Synthesis, vols. 1-17, John Wiley and Sons, New York, N.Y., 1991; Rodd's Chemistry of Carbon Compounds, vols. 1-5 and supps., Elsevier Science Publishers, 1989; Organic Reactions, vols. 1-40, John Wiley and Sons, New York, N.Y., 1991; March J.: Advanced Organic Chemistry, 4th ed., John Wiley and Sons, New York, N.Y.; and Larock: Comprehensive Organic Transformations, VCH Publishers, New York, 1989.

In some cases, protective groups may be introduced and finally removed. Suitable protective groups for amino, hydroxy, and carboxy groups are described in Greene et al., Protective Groups in Organic Synthesis, Second Edition, John Wiley and Sons, New York, 1991. Standard organic chemical reactions can be achieved by using a number of different reagents, for examples, as described in Larock: Comprehensive Organic Transformations, VCH Publishers, New York, 1989.

In one variation, a variety of distinct ligands disclosed in the present application can be synthesized by the general steps outlined in Scheme 1.

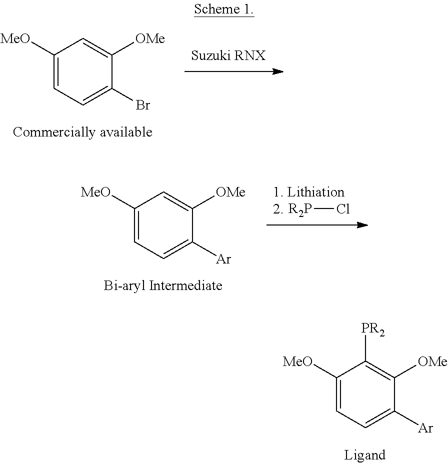

An aryl halide derivative, such as the commercially available compound 1-bromo-2,4-dimethoxybenzene, may be coupled with an aromatic compound under standard Suzuki reaction conditions to provide a bi-aryl intermediate compound. Similarly, the mono-amino analog as well as the 1,3-diaminobenzene analog and its amino-alkyl (—NHC₁₋₁₀alkyl) and amino-dialkyl (—N(C₁₋₁₀alkyl)₂ derivatives, for example, may be used as starting materials to prepare the corresponding amino derivatives and analogs. Regioselective lithiation under standard conditions provides the lithiated aryl that may be coupled with a phosphorous halide, such as R₂P—Cl, to provide the Ligand with the desired functionality, depending on the nature of the starting halide, the aryl coupling partner in the Suzuki reaction, as well as the nature of the phosphorous halide.

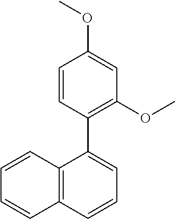 [SMILES: COc1ccc(-c2cccc3ccccc23)c(OC)c1]

(1) 1-(2,4-Dimethoxyphenyl)naphthalene: An oven dried 5 mL microwave vial with a 10 mm stir bar was charged with naphthalene-1-boronic acid (172 mg, 1.0 mmol) and tribasic potassium phosphate monohydrate (288 mg, 1.25 mmol). The vial was equipped with a septum and subjected to 3 evacuation/argon backfill cycles. 2,4-Dimethoxybromobenzene (0.071 mL, 0.5 mmol) was added to the vial via syringe followed by the addition of a toluene solution (0.1 mL) of Pd(OAc)₂ (0.56 mg, 0.0025 mmol) and EvanPhos (1.8 mg, 0.0038 mmol) via syringe. A 2 wt % solution of TPGS-750-M in degassed water (0.9 mL) was added and the reaction was stirred in an oil bath at 40° C. under argon. GC/MS monitoring showed complete consumption of the halide after 90 min. The vessel was cooled to rt and the crude mixture was extracted in flask with EtOAc (3×1 mL). The combined organic phases were flushed through a short plug of silica gel in a pipette and then washed with EtOAc. Volatiles were removed in vacuo. The mixture was chromatographed over silica gel eluting with 1:9 diethyl ether:hexanes (R_{f}=0.42, 1:4 diethyl ether:hexanes) to yield a white powder (116 mg, 88%). ¹H NMR (500 MHz, chloroform-d) δ 7.87 (dt, J=8.1, 0.8 Hz, 1H), 7.84 (dt, J=8.4, 1.1 Hz, 1H), 7.61 (dq, J=8.4, 1.0 Hz, 1H), 7.53-7.49 (m, 1H), 7.47-7.43 (m, 1H), 7.40-7.36 (m, 2H), 7.21-7.18 (m, 1H), 6.64-6.60 (m, 2H), 3.90 (s, 3H), 3.68 (s, 3H). ¹³C NMR (101 MHz, chloroform-d) δ 160.69, 158.30, 136.87, 133.61, 132.58, 132.38, 128.22, 127.68, 127.59, 126.62, 125.68, 125.62, 125.51, 122.33, 104.36, 98.87, 55.67, 55.59.

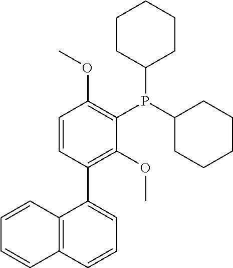 [SMILES: COc1ccc(-c2cccc3ccccc23)c(OC)c1P(C1CCCCC1)C1CCCCC1]

(L1) Dicyclohexyl(2,6-dimethoxy-3-(naphthalen-1-yl)phenyl)phosphane: A flame dried 100 mL 3-neck round bottom flask containing a magnetic stir bar was charged with biaryl 1 (2.0 g, 7.57 mmol) under a flow of argon. The vessel was evacuated and back-filled with argon 3 times. The vessel was charged with anhydrous THF (35 mL) and stirred until dissolution of the biaryl was visually complete. The vessel was submerged in an ice bath and stirred for 10 min. n-Butyllithium (2.35 M in hexanes, 3.07 mL, 7.21 mmol) was added to the stirring solution dropwise via syringe over 15 min. Upon complete addition of n-butyllithium, the solution was stirred in the ice bath for 30 min. and the vessel was removed and stirring continued for another 30 min. The vessel was re-submerged in an ice bath and chlorodicyclohexyl-phosphine (1.55 mL, 7.04 mmol) was added dropwise via syringe over 10 min. The solution was stirred in the ice bath for 30 min. and the vessel was removed from the ice bath. Stirring was continued at rt for 3 h. The solution was quenched with water (30 mL) and diluted with diethyl ether (100 mL). The phases were separated and the aqueous phase was extracted with diethyl ether (3×35 mL). The combined organic phases were washed with a solution of 10% sulfuric acid/water (5×20 mL). The combined acidic phases were mixed with diethyl ether (150 mL) and a saturated solution of sodium carbonate in water was slowly added while gently swirling until gas evolution had ceased. The phases were separated and the aqueous phase was extracted with diethyl ether (2×50 mL). The ether was dried over anhydrous MgSO₄ and concentrated in vacuo yielding a flocculent white solid (3.22 g, 99%). ¹H NMR (400 MHz, chloroform-d) δ 7.90-7.83 (m, 2H), 7.67 (d, J=8.3 Hz, 1H), 7.55-7.27 (m, 5H), 6.73 (d, J=8.4 Hz, 1H), 3.89 (s, 3H), 3.18 (s, 3H), 2.39 (dddt, J=18.4, 11.4, 6.7, 3.4 Hz, 2H), 2.01-1.02 (m, 20H). ¹³C NMR (101 MHz, chloroform-d) δ 163.82, 163.68, 163.13, 137.05, 134.05, 133.56, 132.11, 128.01, 127.53, 127.43, 126.36, 126.16, 125.79, 125.60, 125.30, 105.95, 61.15, 55.48, 34.90-34.10 (m), 32.59, 32.35, 31.10-30.22 (m), 27.62-26.95 (m). ³¹P NMR (162 MHz, chloroform-d) δ-9.22.

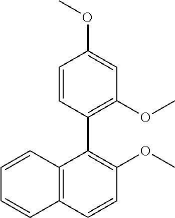 [SMILES: COc1ccc(-c2c(OC)ccc3ccccc23)c(OC)c1]

(2) 1-(2,4-Dimethoxyphenyl)-2-methoxynaphthalene: A flame dried 250 mL 3-neck round bottomed flask containing a magnetic stir bar was charged with 2-methoxybromonaphthalene (3.56 g, 15.0 mmol), 2,4-dimethoxyphenylboronic acid (5.46 g, 30.0 mmol), and tribasic potassium phosphate monohydrate (8.64 g, 37.5 mmol) under a flow of argon. The vessel was evacuated and backfilled with argon 3 times. A solution of Pd(OAc) (33.7 mg, 0.15 mmol) and L1 (0.104 g, 0.225 mmol) in THF (1.5 mL) was added via syringe to the vessel followed by THF (1.5 mL) and a degassed solution of 2 wt % TPGS-750-M in water (27 mL). The vessel was placed in an oil bath at 35° C. and stirred vigorously. GC/MS and TLC monitoring showed complete consumption of the bromide after 4 h. The crude mixture was transferred to a separatory funnel and extracted with EtOAc (3×20 mL). The combined organic phases were dried over anhydrous Na₂SO₄ followed by solvent removal in vacuo. The mixture was chromatographed on silica gel eluting with 1:4 diethyl ether:hexanes (R_{f}=0.20 1:4 diethyl ether:hexanes). The pure product was collected. The impure fractions were collected separately, concentrated and the product was recrystallized from a 1:1 mixture of EtOAc:hexanes yielding an off-white/gray powder (combined 1.54 g, 70%). ¹H NMR (500 MHz, chloroform-d) δ 7.87 (d, J=9.0 Hz, 1H), 7.83-7.79 (m, 1H), 7.44-7.40 (m, 1H), 7.37 (d, J=9.0 Hz, 1H), 7.34-7.29 (m, 2H), 7.13 (d, J=7.8 Hz, 1H), 6.67-6.63 (m, 2H), 3.90 (s, 3H), 3.85 (s, 3H), 3.68 (s, 3H). ¹³C NMR (101 MHz, chloroform-d) δ 160.38, 158.63, 154.47, 133.97, 132.63, 129.04, 128.90, 127.80, 126.03, 125.31, 123.33, 121.77, 117.67, 114.10, 104.40, 99.03, 56.93, 55.66, 55.34.

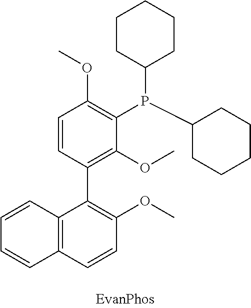 [SMILES: COc1ccc(-c2c(OC)ccc3ccccc23)c(OC)c1P(C1CCCCC1)C1CCCCC1]

Dicyclohexyl(2,6-dimethoxy-3-(2-methoxynaphthalen-1-yl)phenyl)phosphane (EvanPhos): A flame dried 100 mL 3-neck round bottom flask containing a magnetic stir bar was charged with biaryl 2 (1.70 g, 5.78 mmol) under a flow of argon. The vessel was evacuated and back-filled with argon 3 times. The vessel was charged with anhydrous THF (30 mL) and stirred until dissolution of the biaryl was visually complete. The vessel was submerged in an ice bath and stirred for 10 min. n-Butyllithium (2.45 M in hexanes, 2.25 mL, 5.52 mmol) was added to the stirring solution dropwise via syringe over 10 min. Upon complete addition of n-butyllithium, the solution stirred in the ice bath for 30 min. and the vessel was removed and stirring continued for another 30 min. The vessel was re-submerged in an ice bath and chlorodicyclohexylphosphine (1.16 mL, 5.26 mmol) was added dropwise via syringe over 10 min. The solution was stirred in the ice bath for 30 min., and the vessel was removed from the ice bath. Stirring was continued at rt for 12 h. The solution was quenched with water (25 mL) and diluted with diethyl ether (100 mL). The phases were separated and the aqueous phase was extracted with diethyl ether (2×50 mL). The combined organic phases were washed with a solution of 10% sulfuric acid/water (5×15 mL). The combined acidic phases were mixed with diethyl ether (150 mL) and a saturated solution of sodium carbonate in water was slowly added while gently swirling until gas evolution had ceased. The phases were separated and the aqueous phase was extracted with diethyl ether (2×50 mL). The ether was dried over anhydrous MgSO₄ and concentrated in vacuo. The product was purified by chromatography over basic alumina eluting with 1:3 diethyl ether:hexanes (R_{f}=0.26 1:3 diethyl ether:hexanes) which yielded a flocculent white solid (1.54 g, 58%). ¹H NMR (400 MHz, chloroform-d) δ 7.88 (d, J=9.0 Hz, 1H), 7.83-7.79 (m, 1H), 7.48-7.43 (m, 1H), 7.39-7.29 (m, 3H), 7.18 (d, J=8.3 Hz, 1H), 6.75 (d, J=8.4 Hz, 1H), 3.88 (s, 3H), 3.87 (s, 3H), 3.22 (s, 3H), 2.46-2.30 (m, 2H), 2.01-1.88 (m, 2H), 1.81-1.54 (m, 8H), 1.37-1.06 (m, 10H). ¹³C NMR (101 MHz, chloroform-d) δ 164.78, 164.65, 163.30, 154.43, 134.41, 133.87, 129.17, 129.14, 127.95, 126.42, 125.39, 123.56, 122.56, 121.98, 113.93, 106.20, 61.05, 56.90, 55.58, 34.76 (d, J=11.5 Hz), 34.32 (d, J=11.7 Hz), 32.71 (d, J=24.5 Hz), 32.34 (d, J=22.2 Hz), 30.70 (dd, J=8.8, 6.4 Hz), 27.64-27.15 (m), 26.67. ³¹P NMR (162 MHz, chloroform-d) δ-9.24.

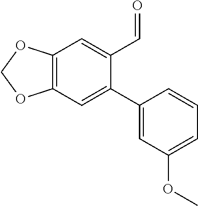 [SMILES: COc1cccc(-c2cc3c(cc2C=O)OCO3)c1]

6-(3-Methoxyphenyl)benzo[d][1,3]dioxole-5-carbaldehyde: An oven dried 5 mL microwave vial with a 10 mm stir bar was charged with 6-bromopiperonal (115 mg, 0.5 mmol), 3-methoxybenzeneboronic acid (152 mg, 1.0 mmol) and tribasic potassium phosphate monohydrate (288 mg, 1.25 mmol). The vial was equipped with a septum and subjected to three evacuation/argon backfill cycles. A toluene solution (0.1 mL) of Pd(OAc)₂ (0.56 mg, 0.0025 mmol) and EvanPhos (1.8 mg, 0.0038 mmol) was added via syringe followed by toluene (0.9 mL). The reaction was stirred in an oil bath at 40° C. under an argon atmosphere. GC/MS monitoring showed complete consumption of the halide after 7 h. The vessel was cooled to rt and diluted with water (1 mL). The aqueous phase was extracted in flask with EtOAc (3×1 mL). The combined organic phases were flushed over a short plug of silica gel in a pipette and washed with EtOAc. Volatiles were removed in vacuo. The mixture was chromatographed over silica gel eluting with 1:4 diethyl ether:hexanes (R_{f}=0.25, 1:4 diethyl ether:hexanes) which yielded a colorless viscous oil (119 mg, 93%). ¹H NMR (400 MHz, chloroform-d) δ 9.76 (s, 1H), 7.45 (s, 1H), 7.34 (t, J=7.9 Hz, 1H), 6.96 (dd, J=8.3, 2.5 Hz, 1H), 6.90 (d, J=7.8 Hz, 1H), 6.88-6.86 (m, 1H), 6.84 (s, 1H), 6.08 (s, 2H), 3.83 (s, 3H). ¹³C NMR (101 MHz, chloroform-d) δ 190.75, 159.50, 152.10, 147.88, 143.54, 138.97, 129.44, 128.89, 122.82, 115.83, 113.67, 110.21, 110.11, 106.25, 102.21, 66.66, 55.43.

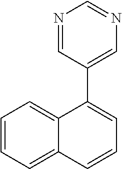 [SMILES: c1ccc2c(-c3cncnc3)cccc2c1]

5-(Naphthalen-1-yl)pyrimidine: An oven dried 5 mL microwave vial with a 10 mm stir bar was charged with 5-bromopyrimidine (115 mg, 0.5 mmol), naphthalene-1-boronic acid (152 mg, 1.0 mmol) and tribasic potassium phosphate monohydrate (288 mg, 1.25 mmol). The vial was equipped with a septum and subjected to three evacuation/Argon backfill cycles. A toluene solution (0.1 mL) of Pd(OAc)₂ (0.56 mg, 0.0025 mmol) and EvanPhos (1.8 mg, 0.0038 mmol) was added via syringe followed by toluene (0.9 mL). The reaction was stirred in an oil bath at 40° C. under argon. GC/MS monitoring showed complete consumption of the halide after 6 h. The vessel was cooled to rt and diluted with water (1 mL). The aqueous phase was extracted in flask with EtOAc (3×1 mL). The combined organic phases were flushed over a short plug of silica gel in a pasteur pipette and then washed with EtOAc. Volatiles were removed in vacuo. The mixture was chromatographed over silica gel eluting with 1:3 diethyl ether:hexanes (R_{f}=0.20, 3:7 diethyl ether:hexanes) which yielded an off-white powder (99 mg, 96%). ¹H NMR (400 MHz, chloroform-d) δ 9.31 (s, 1H), 8.89 (s, 2H), 7.95 (dd, J=8.4, 3.7 Hz, 2H), 7.75 (d, J=8.2 Hz, 1H), 7.61-7.48 (m, 3H), 7.42 (d, J=7.0 Hz, 1H). ¹³C NMR (101 MHz, chloroform-d) δ 157.72, 157.40, 134.44, 133.87, 132.50, 131.26, 130.95, 129.53, 128.78, 127.84, 127.18, 126.54, 125.52, 124.65.

Results from Representative Coupling Reactions:

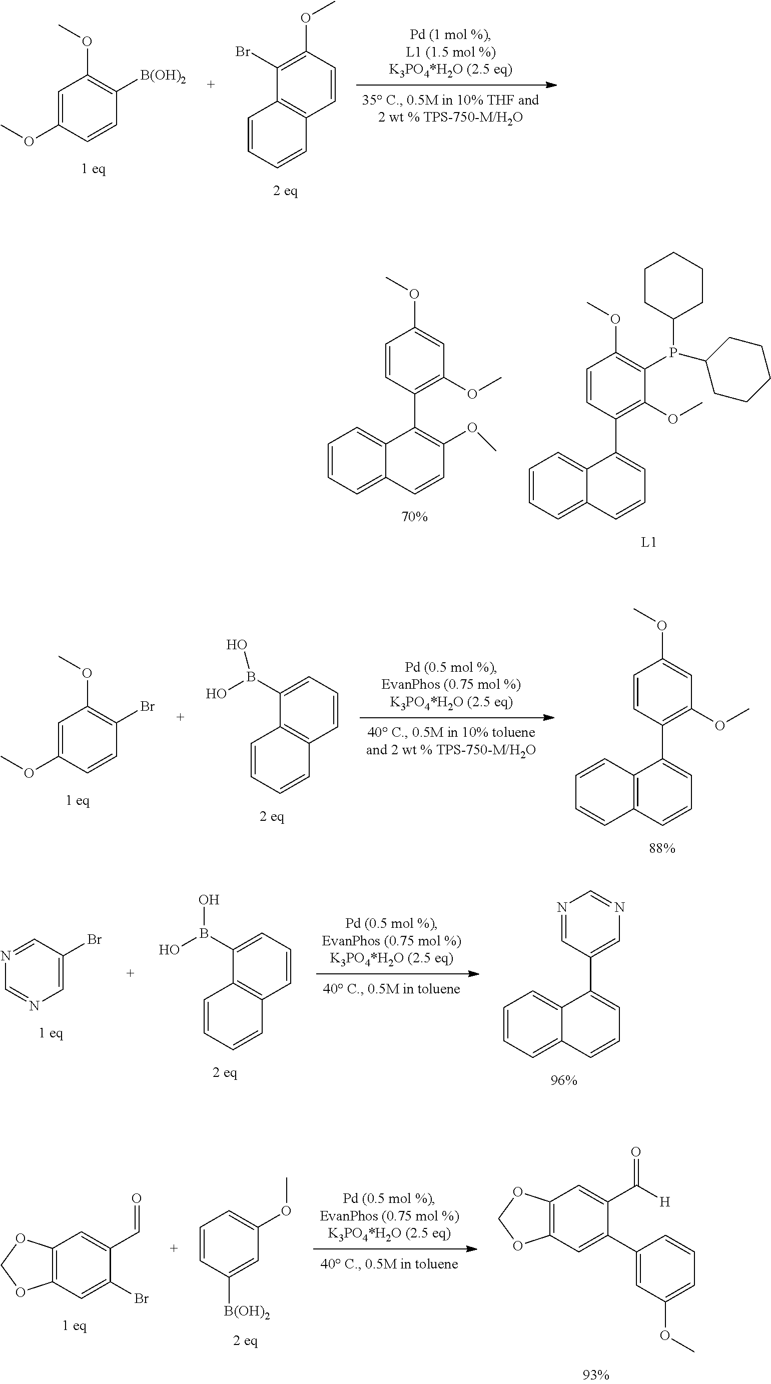

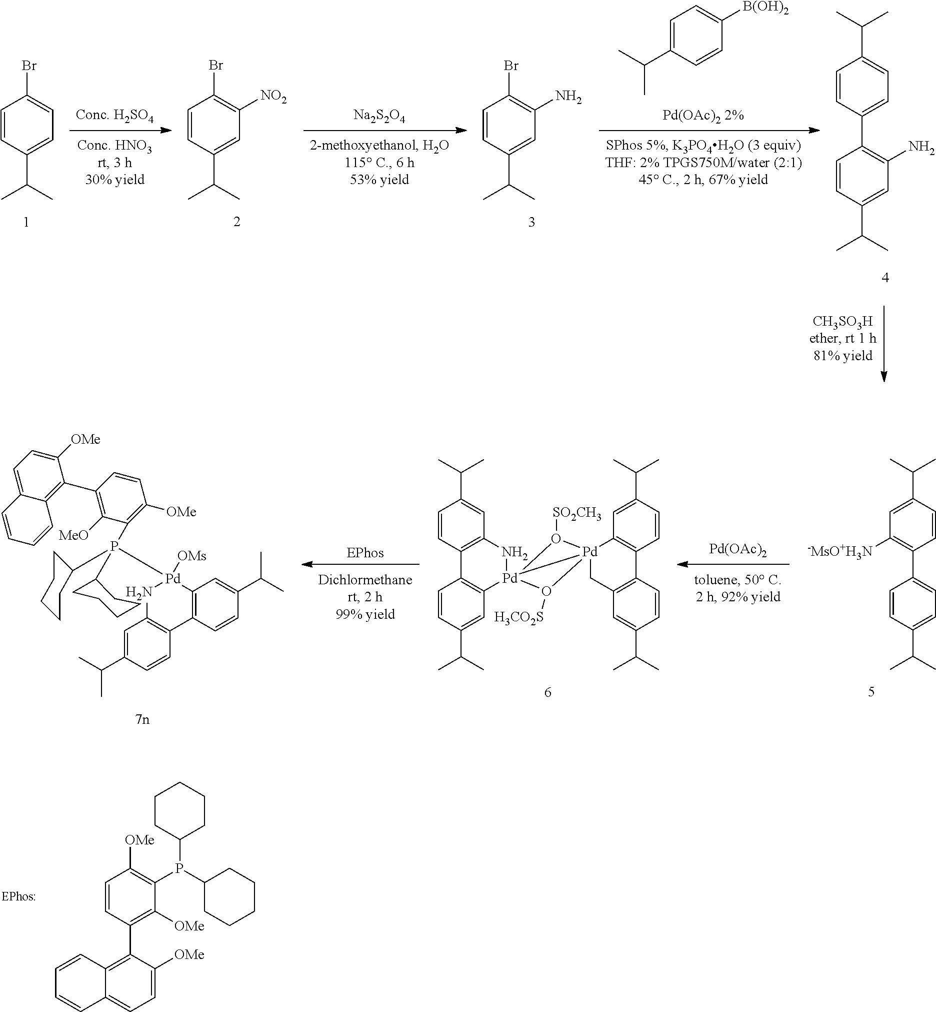

1-Bromo-4-isopropyl-2-nitrobenzene (2): Nitrating mixture was prepared by adding concentrated sulphuric acid (72.3 mmol, 3.9 mL) to nitric acid (48.4 mmol, 3.0 mL) at 0° C. This nitrating mixture was added to 1-bromo-4-isopropylbenzene 1 (32.3 mmol, 6.4 g) dropwise by maintaining temperature to 0° C. The mixture was stirred at RT for 5 h before pouring it on ice water. The aqueous reaction mixture was extracted with ethyl acetate (3×15 mL). Evaporation of ethyl acetate lead to crude yellow oil. This oil was then purified using chromatography on SiO₂ with eluent ethyl acetate:hexanes (0 to 5%). The product was pale yellow oil at RT and freezes upon cooling (30%, 2.3 g).

2-Bromo-5-isopropylaniline (3): 1-Bromo-4-isopropyl-2-nitrobenzene 2 (4.76 mmol, 1.23 g) and sodium dithionate (17.6 mmol, 3.0 g) was added to mixture of glycol monomethylether:water (1:1) 15 mL at room temperature and this reaction mixture was refluxed at 115° C. for 6 hours. The reaction mixture was cooled to room temperature and 50% HCl was added to this mixture and refluxed again for 20 min. The reaction mixture was poured on ice water, neutralized with solid Na₂CO₃, and extracted with ethyl acetate (3×20 mL). Evaporation of ethyl acetate lead to crude yellow oil. This oil was then purified using chromatography on SiO₂ with eluent ethyl acetate:hexanes (0 to 3%). The product was yellow oil (53%, 504 mg).

4,4′-Diisopropyl-[1,1′-biphenyl]-2-amine (4): 2-Bromo-5-isopropylaniline 3 (1.1 mmol, 237 mg), 4-isopropylphenyl boronic acid (1.43 mmol, 236 mg), K₃PO₄.H₂O (3.3 mmol, 753 mg), SPhos (5 mol %, 0.055 mmol, 22.6 mg), Pd(OAc)₂ (2 mol %, 0.022 mmol, 4.9 mg) was added to 1 dram screw cap vial with rubber septum. The vial was degassed and backfilled with argon (this procedure was repeated 3 times). Three mL of 2% TPGS-750-M:THF (1:2) was added and stirred at 45° C. for 6 h. The reaction mixture was extracted with ethyl acetate (3×1 mL). Evaporation of ethyl acetate lead to crude oil, which was then purified using chromatography on SiO₂ with eluent ethyl acetate:hexanes (0 to 5%). The product was dark yellow oil (67%, 188 mg).

Representative procedure to prepare 4,4′-diisopropyl-[1,1′-biphenyl]-2-amine mesylate salt (5):

In a two-necked round-bottomed flask, 4,4′-diisopropyl-[1,1′-biphenyl]-2-amine (4) (0.29 mmol, 75 mg) was dissolved in 2 ml. anhydrous diethyl ether. Methanesulfonic acid (0.29 mmol, 28.4 mg) was slowly added to the reaction mixture. Reaction mixture was stirred at RT for additional 30 min. Appearance of white solid suspension in a reaction mixture was indicative of salt formation. Solid was filtered through a frit and washed with additional 3 ml cold ether. Solid was dried under reduced pressure to obtain pure compound as white solid (81%, 81.2 mg).

Representative procedure to prepare μ-OMs dimer 4,4′-diisopropyl-[1,1′-biphenyl]-2-amine mesylate (6):

4,4′-Diisopropyl-[1,1′-biphenyl]-2-amine mesylate (81.2 mg, 0.232 mmol) and palladium acetate (52.2 mg, 0.232 mmol) were transferred in to a sealable reaction vessel. Reaction vessel was evacuated and backfilled with argon for couple of times. Reaction vessel was opened under the counter-flow of argon, and 2 ml anhydrous toluene was added via syringe. The mixture was stirred at 50° C. for 2 h. The appearance of solid suspension was the indicative of complex formation. Reaction mixture was cooled to RT, and solid was filtered through a frit. Resulting solid was washed with addition 10 ml hexanes to obtain pure compound as brown solid (92%, 194 mg).

Similarly, mesylate salts of other biarylamines were prepared. In addition to mesylates, other sulfonates such as ethanesulfonate, benzenesulfonate, p-toluenesulfonate, trifluoromethane sulfonate, camphosulfonate may also be used in the disclosed process.

Representative procedure to prepare EphosPdCycle of 4,4′-diisopropyl-[1,1′-biphenyl]-2-amine mesylate (7n):

μ-OMs dimer 4,4′-diisopropyl-[1,1′-biphenyl]-2-amine mesylate (28 mg, 0.03 mmol) and Ephos (29.4 mg, 0.06 mmol) were transferred to a sealable reaction vessel. Reaction vessel was sealed, evacuated and backfilled with argon by at least three times. Reaction vessel was opened under the counter-flow of argon, and 2 ml dry CH₂Cl₂ was added to it via syringe. The mixture was stirred at RT under argon flow for 2 h. Solvent was evaporated under reduced pressure to obtain solid. Resulting solid was washed several times with dry pentanes to obtain pure complex a reddish brown solid (99%, 56.6 mg).

Representative palladacycles, such as EvanPhos-PdCycle, may be prepared according to the disclosed procedures.

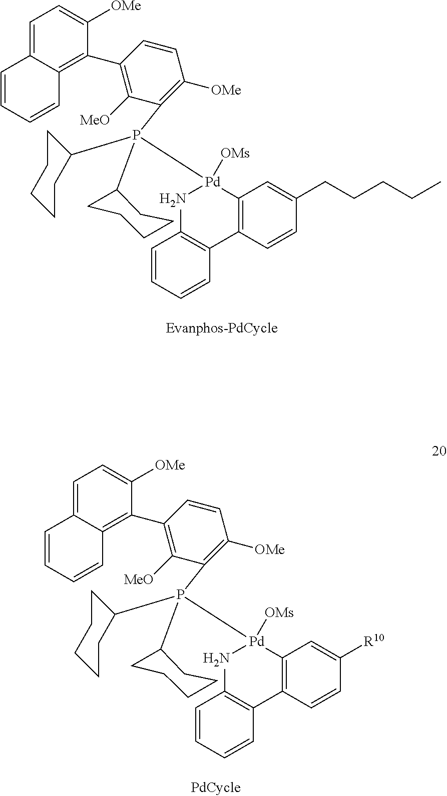

wherein R¹⁰ is H or is selected from the group consisting of —OC₁₋₁₀alkyl, C₁₋₁₀alkyl, —SR⁸, —NR⁸R⁹, C₆₋₁₄aryl and C₄₋₁₂heteroaryl.

Similarly, the following palladacycles were prepared:

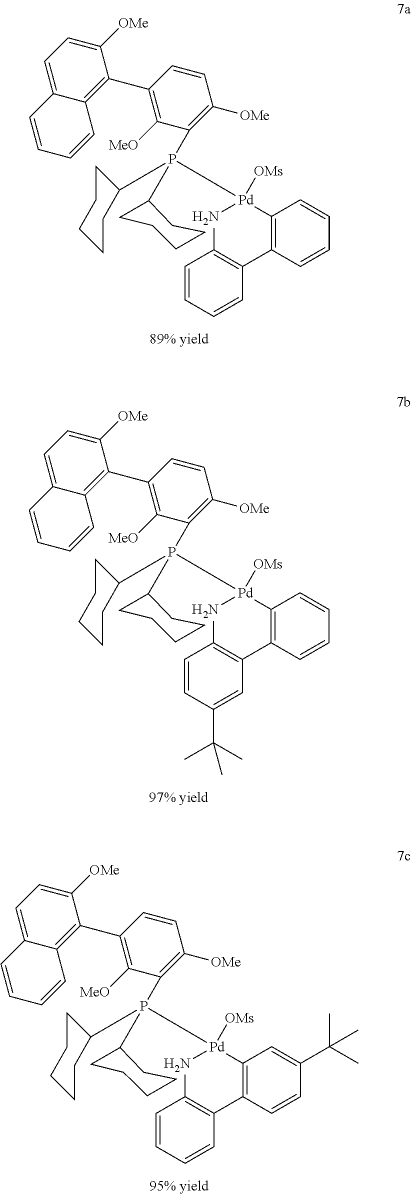

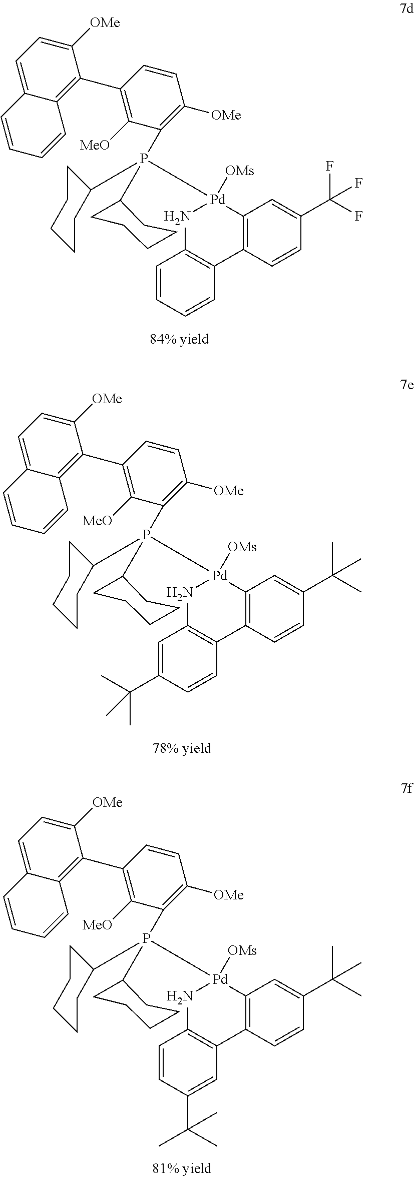

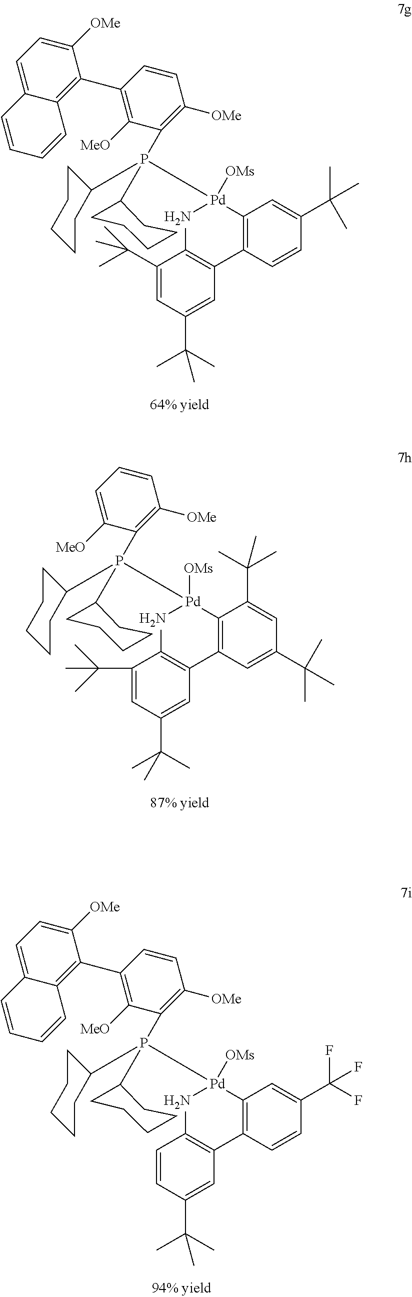

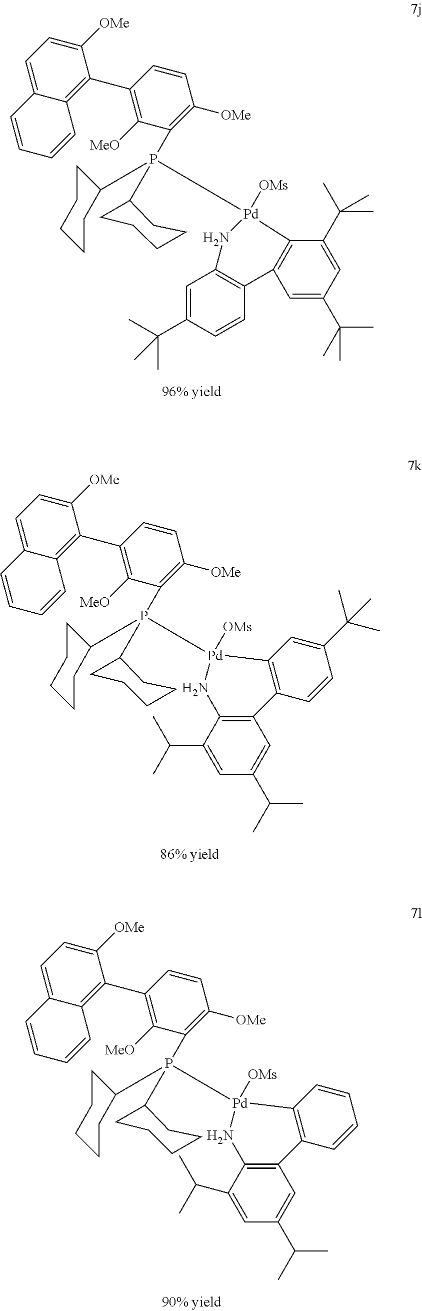

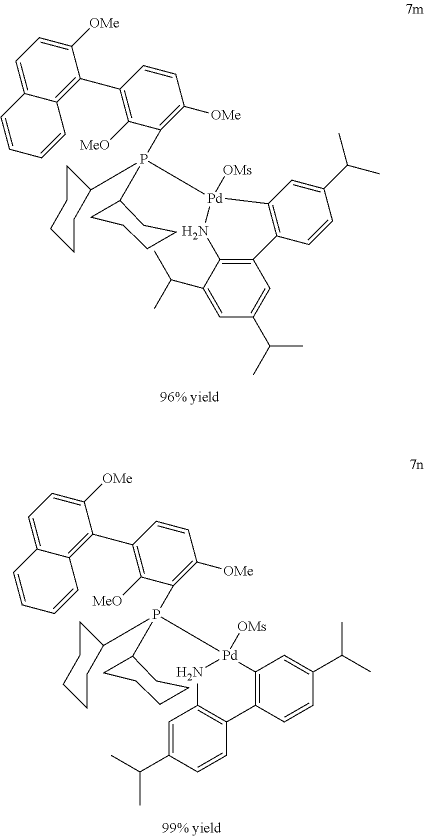

### Preparation of Stock Solution of Palladacycle 7n:

Add 14.4 mg of the palladacycle 7n (molecular weight: 944.5 g/mol) in a glass vial and dissolve it in 1.0 mL of dry dichloromethane to get clear solution after stirring for about 2 minutes. This solution corresponds to 30,000 ppm of Pd.

### Procedure for Suzuki-Miyuara Reaction:

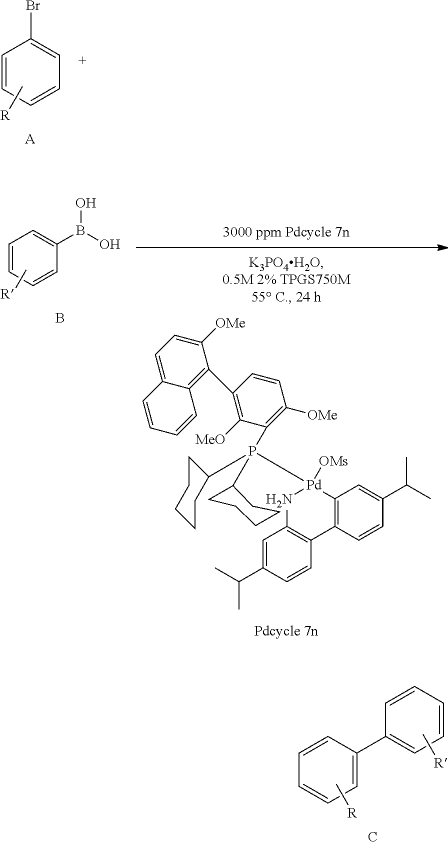

Procedure for the preparation of the analogous aminonapthyl analog of EvanPhos: In a 1 dram screw cap vial, 100 μl (3000 ppm Pd) solution of above stock solution was added. Dichloromethane was then evacuated to dryness for about 1 h under high vacuum. To this vial, aryl halide A (0.5 mmol), boronic acid B (0.75 mmol) and K₃PO₄.H₂O (0.75 mmol) was added, and it was sealed with a rubber septum. Vial was evacuated and backfilled with argon for three times before adding 1.0 mL of 2% TPGS-750-M under positive flow argon. The rubber septum was quickly replaced with screw cap and reaction was stirred at 55° C. until the completion (as monitored by GC-MS or TLC).

### Procedure for Suzuki Coupling Reactions:

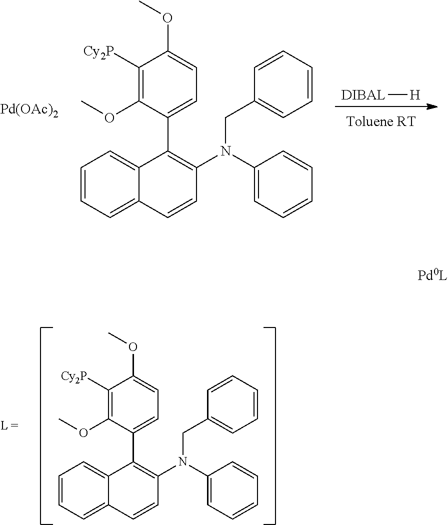

Pd(OAc)₂ (4.5 mg, 0.02 mmol), and an aminonaphthyl analog to EvanPhos Ligand, above, (25.7 mg, 0.04 mmol) are dissolved in 2 mL of toluene and set to stir for about 15 min in a microwave vial under a bed of argon. A solution of DIBAL-H in DCM (0.1 mL, 1.0 M solution) is added to the solution dropwise and set to stir for an additional 10 min. A color change is observed. The solution turned from a yellow/gold color to a dark brown/black color indicating the formation of Pd⁰.

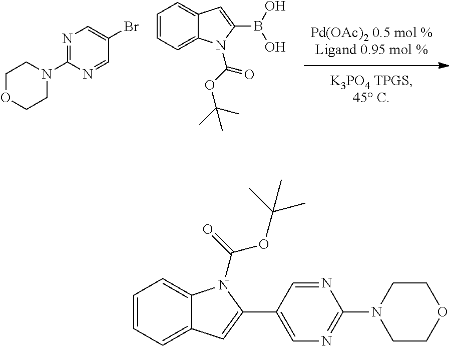

A 1 dram vial was charged with aryl bromide (122 mg, 0.5 mmol), aryl boronic acid (196 mg, 0.75 mmol) and K₃PO₄ (172 mg, 0.75 mmol). 2 wt % TPGS is added to the vial (1 mL) and the mixture is set to stir for about 5 min. The catalyst solution is added to the reaction mixture (0.25 mL, 0.5 mol % Pd) and the reaction vessel is purged with argon, sealed with Teflon and set to stir for 1.5 hrs at 45° C.

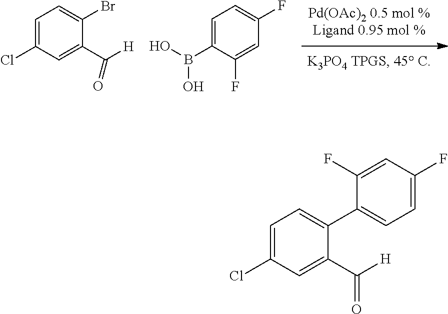

Following the general procedure from above, the aryl bromide (110 mg, 0.5 mmol), aryl boronic acid (118 mg, 0.75 mmol), K₃PO₄ (172 mg, 0.75 mmol), 2 wt % TPGS (1 mL), and the catalyst solution (0.25 mL). Reaction was stirred for 3 hours at 45° C.

While a number of exemplary embodiments, aspects and variations have been provided herein, those of skill in the art will recognize certain modifications, permutations, additions and combinations and certain sub-combinations of the embodiments, aspects and variations. It is intended that the following claims are interpreted to include all such modifications, permutations, additions and combinations and certain sub-combinations of the embodiments, aspects and variations are within their scope. The entire disclosures of all documents cited throughout this application are incorporated herein by reference.

### REFERENCES

[1] a) C. C. C. J Seechurn et al., Angew. Chem., Int. Ed. 2012, 51, 5062-5085; b) H. Li et al., ACS Catal. 2012, 2, 1147-1164.
[2] a) S. Z. Tasker et al., Nature 2014, 509, 299-309; b) S. D. Ramgren et al., Org. Lett. 2013, 15, 3950-3953; c) F.-S. Han, Chem. Soc. Rev. 2013, 42, 5270-5298; d) F. Gonzalez-Bobes et al., J. Am. Chem. Soc. 2006, 128, 5360-5361; e) L. Hie et al., J. Chem. Educ. 2014; f) A. H. Christian et al., Organometallics 2014, 33, 2134-2137; g) L. Chen et al., Eur. J. Org. Chem. 2014, 2014, 4953-4957; h) X. Wu et al., J. Am. Chem. Soc. 2014, 136, 1789-1792; i) J. C. Tellis et al., Science 2014, 345, 433-436.
[3] a) S. K. Gurung et al., Org. Lett. 2014, 16, 1264-1267; b) Y. Zhou et al., Angew. Chem., Int. Ed. 2014, 126, 3543-3547; c) N. He et al., Org. Lett. 2015; d) L. Cheng et al., RSC Adv. 2014, 4, 44312-44316; e) Y.-Y. Sun et al, Chem. Commun. 2014, 50, 11060-11062; f) C.-T. Yang et al., Angew. Chem., Int. Ed. 2011, 50, 3904-3907; g) J. Mao et al., Tetrahedron 2008, 64, 3905-3911.
[4] a) N. Zhang et al., J. Org. Chem. 2012, 77, 5956-5964; b) M. B. Thathagar et al., J. Am. Chem. Soc. 2002, 124, 11858-11859; c) M. R. Netherton et al., Adv. Synth. Catal. 2004, 346, 1525-1532; d) G. D. Allred et al., J. Am. Chem. Soc. 1996, 118, 2748-2749; e) L. Xu et al., Org. Lett. 2010, 12, 884-887.

What is claimed is:

1. A ligand of the formula A: 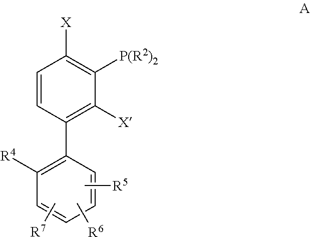
wherein:
X is selected from —OR¹ or —NR′R″ where R′ and R″ is independently selected from the group consisting of H, C₁₋₁₀alkyl, C₃₋₆cycloalkyl, C₆₋₁₄aryl and C₄₋₁₂heteroaryl;
X′ is selected from —OR³ or —NR′R″ where R′ and R″ is independently selected from the group consisting of H, C₁₋₁₀alkyl, C₃₋₆cycloalkyl, C₆₋₁₄aryl and C₄₋₁₂heteroaryl;
each R¹ and R³ is independently selected from a group consisting of C₁₋₁₀alkyl, C₃₋₆cycloalkyl, C₆₋₁₄aryl and C₄₋₁₂heteroaryl;
R² is selected from the group consisting of C₁₋₁₀alkyl, C₃₋₆cycloalkyl, C₆₋₁₄aryl and substituted C₆₋₁₄aryl and C₄₋₁₂heteroaryl;
R⁴ is H or is selected from the group consisting of —OC₁₋₁₀alkyl, C₁₋₁₀alkyl, C₃₋₆cycloalkyl, —SR⁸, —NR⁸R⁹, C₆₋₁₄aryl and C₄₋₁₂heteroaryl;
each R⁵ and R⁶ is H or R⁵ and R⁶ together with the aryl group to which they are attached to form a fused substituted or unsubstituted aromatic ring or heteroaromatic ring;
R⁷ is H or is selected from the group consisting of —OC₁₋₁₀alkyl, C₁₋₁₀alkyl, —SR⁸, —NR⁸R⁹, C₆₋₁₄aryl and C₄₋₁₂heteroaryl; and
each R⁸ and R⁹ is independently H or C₁₋₁₀alkyl.

2. The ligand of claim 1, wherein the ligand is of the formula A-1: 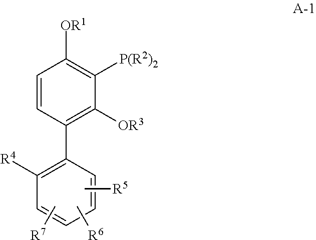
wherein:
each R¹ and R³ is independently selected from a group consisting of C₁₋₁₀alkyl, C₃₋₆cycloalkyl, C₆₋₁₄aryl and C₄₋₁₂heteroaryl;
R² is selected from the group consisting of C₁₋₁₀alkyl, C₃₋₆cycloalkyl, C₆₋₁₄aryl and substituted C₆₋₁₄aryl and C₄₋₁₂heteroaryl;
R⁴ is H or is selected from —OC₁₋₁₀alkyl and C₃₋₆cycloalkyl;
each R⁵ and R⁶ is H or R⁵ and R⁶ are each independently an aryl or a heteroaryl ring, or R⁵ and R⁶ together with the aryl group to which they are attached to form a substituted or unsubstituted aromatic ring; and
R⁷ is H or is selected from the group consisting of —OC₁₋₁₀alkyl, C₁₋₁₀alkyl, —SR⁸, —NR⁸R⁹, C₆₋₁₄aryl and C₄₋₁₂heteroaryl.

3. The ligand of claim 1, wherein R⁵ and R⁶ together form a substituted or unsubstituted aromatic ring or a substituted or unsubstituted heteroaromatic ring.

4. The ligand of claim 1, wherein the ligand is of the formula B or C: 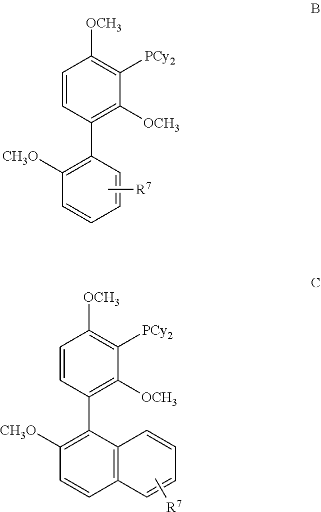
wherein:
R⁷ is H or is selected from the group consisting of —OC₁₋₁₀alkyl, C₁₋₁₀alkyl, —SR⁸, —NR⁸R⁹, C₆₋₁₄aryl and C₄₋₁₂heteroaryl.

5. The ligand of claim 1 selected from the group consisting of A-2 to A-31:

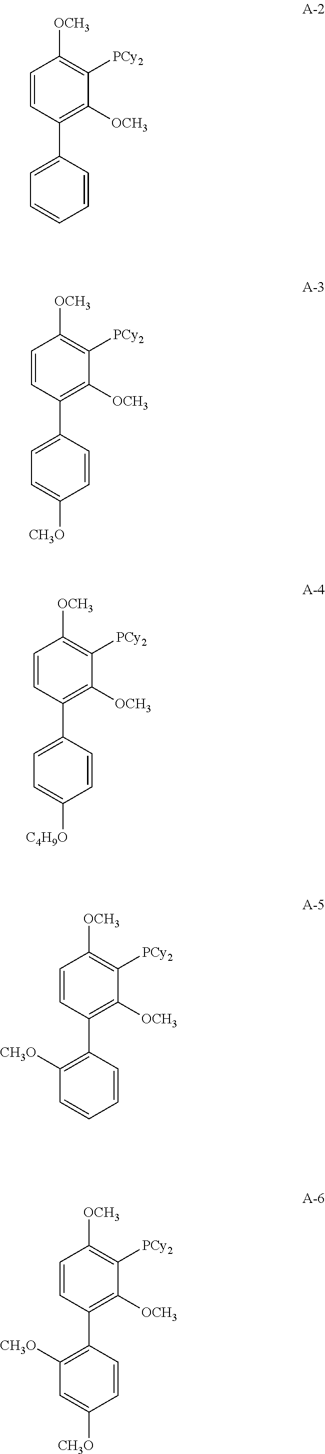

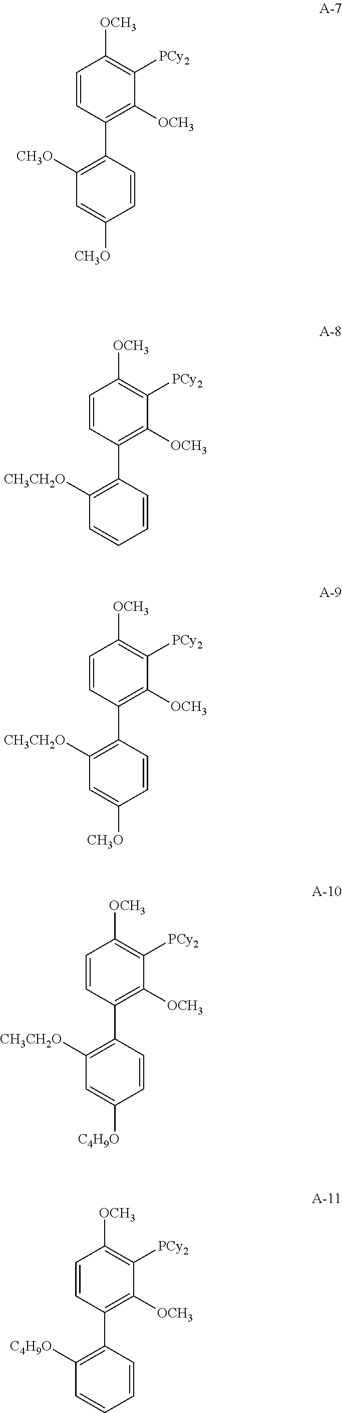

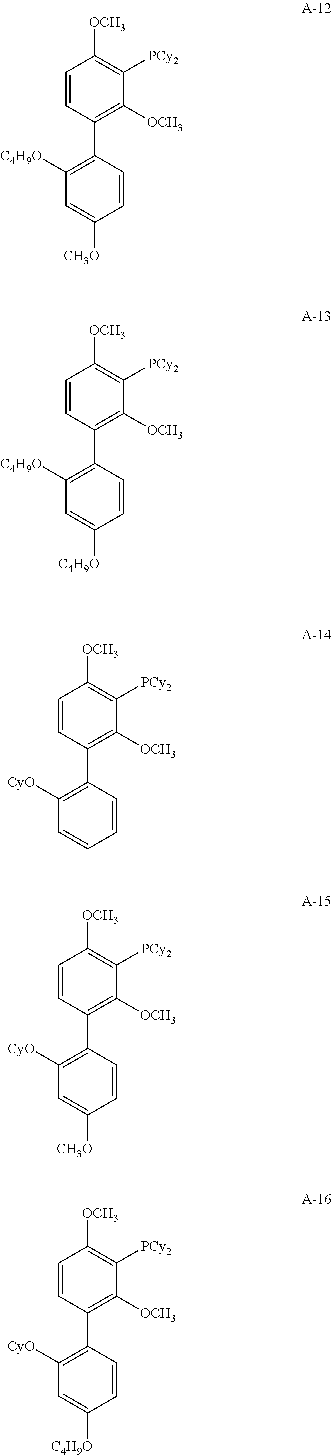

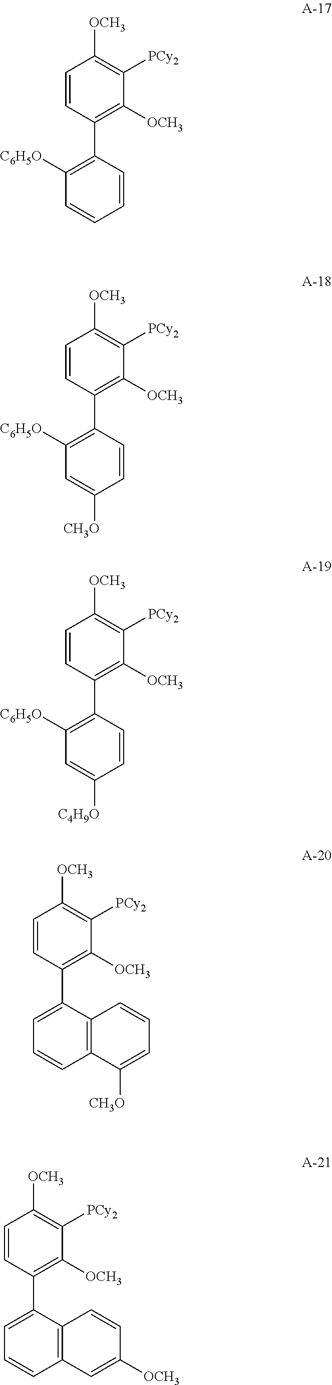

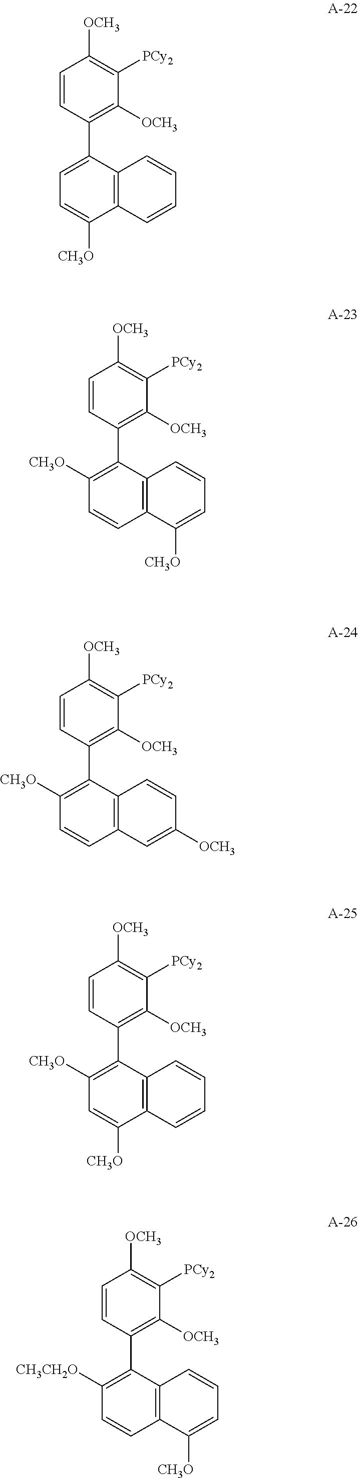

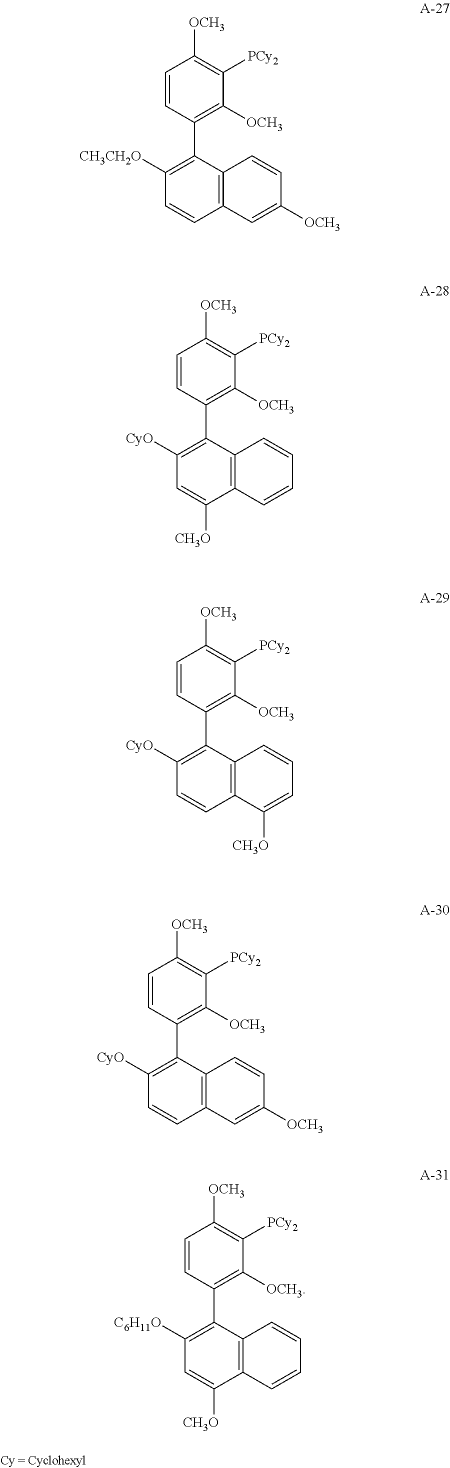

6. A catalyst composition comprising:
a) organometallic nanoparticles comprising:
a nanoparticle (NP) catalyst, prepared by a reduction of an iron salt in an organic solvent, wherein the catalyst comprises at least one other metal selected from the group consisting of Pd, Pt, Au, Ni, Co, Cu, Mn, Rh, Ir, Ru and Os or mixtures thereof;
b) a ligand of the formula A: 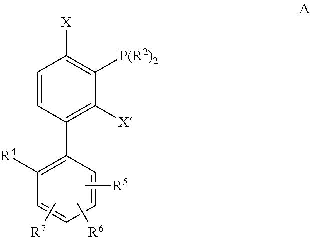
wherein:
X is selected from —OR¹ or —NR′R″ where R′ and R″ is independently selected from the group consisting of H, C₁₋₁₀alkyl, C₃₋₆cycloalkyl, C₆₋₁₄aryl and C₄₋₁₂heteroaryl;
X′ is selected from —OR³ or —NR′R″ where R′ and R″ is independently selected from the group consisting of H, C₁₋₁₀alkyl, C₃₋₆cycloalkyl, C₆₋₁₄aryl and C₄₋₁₂heteroaryl;
each R¹ and R³ is independently selected from a group consisting of C₁₋₁₀alkyl, C₃₋₆cycloalkyl, C₆₋₁₄aryl and C₄₋₁₂heteroaryl;
R² is selected from the group consisting of C₁₋₁₀alkyl, C₃₋₆cycloalkyl, C₆₋₁₄aryl and substituted C₆₋₁₄aryl and C₄₋₁₂heteroaryl;
R⁴ is H or is selected from the group consisting of —OC₁₋₁₀alkyl, C₁₋₁₀alkyl, —SR⁸, —NR⁸R⁹, C₆₋₁₄aryl and C₄₋₁₂heteroaryl;
each R⁵ and R⁶ is H or R⁵ and R⁶ together with the aryl group to which they are attached to form a substituted or unsubstituted aromatic ring or heteroaromatic ring;
R⁷ is H or is selected from the group consisting of —OC₁₋₁₀alkyl, C₁₋₁₀alkyl, —SR⁸, —NR⁸R⁹, C₆₋₁₄aryl and C₄₋₁₂heteroaryl; and
each R⁸ and R⁹ is independently H or C₁₋₁₀alkyl;
c) a surfactant; and
d) a reaction solvent or a reaction medium;
wherein the metal or mixtures thereof is present in less than or equal to 50,000 ppm relative to the iron salt.

7. An aqueous micellar composition for enabling cross-coupling reactions containing organometallic nanoparticles (NPs) as catalyst, comprising:
a) an element selected from the group consisting of Fe, C, H, O, Mg, and a halide, or the entire combination thereof; and
b) palladium, or at least one other metal selected from the group consisting of Pt, Au, Ni, Co, Cu and Mn, or a mixture thereof; wherein the catalyst (NPs) is prepared from a reduction of an iron salt in a solvent and in the presence of a ligand using a reducing agent;
wherein the ligand is of the formula A: 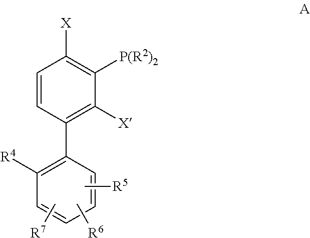
wherein:
X is selected from —OR¹ or —NR′R″ where R′ and R″ is independently selected from the group consisting of H, C₁₋₁₀alkyl, C₃₋₆cycloalkyl, C₆₋₁₄aryl and C₄₋₁₂heteroaryl;
X′ is selected from —OR³ or —NR′R″ where R′ and R″ is independently selected from the group consisting of H, C₁₋₁₀alkyl, C₃₋₆cycloalkyl, C₆₋₁₄aryl and C₄₋₁₂heteroaryl;
each R¹ and R³ is independently selected from a group consisting of C₁₋₁₀alkyl, C₃₋₆cycloalkyl, C₆₋₁₄aryl and C₄₋₁₂heteroaryl;
R² is selected from the group consisting of C₁₋₁₀alkyl, C₃₋₆cycloalkyl, C₆₋₁₄aryl and substituted C₆₋₁₄aryl and C₄₋₁₂heteroaryl;
R⁴ is H or is selected from the group consisting of —OC₁₋₁₀alkyl, C₁₋₁₀alkyl, C₃₋₆cycloalkyl, —SR⁸, —NR⁸R⁹, C₆₋₁₄aryl and C₄₋₁₂heteroaryl;
each R⁵ and R⁶ is H or R⁵ and R⁶ together with the aryl group to which they are attached to form a substituted or unsubstituted aromatic ring or heteroaromatic ring;
R⁷ is H or is selected from the group consisting of —OC₁₋₁₀alkyl, C₁₋₁₀alkyl, —SR⁸, —NR⁸R⁹, C₆₋₁₄aryl and C₄₋₁₂heteroaryl; and
each R⁸ and R⁹ is independently H or C₁₋₁₀alkyl.

8. The composition of claim 6, wherein the ligand is of the formula D: 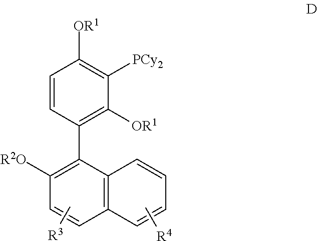
wherein:
each R¹ and R² is independently a C₁₋₃alkyl;
R³ is absent or is selected from the group consisting of —OC₁₋₃alkyl, C₁₋₆alkyl and C₃₋₆cycloalkyl; and
R⁴ is absent or is selected from the group consisting of —OC₁₋₃alkyl and C₁₋₆alkyl.

9. The composition of claim 6, wherein the iron is selected from the group consisting of a Fe(II) or Fe(III) salt, a Fe(II) salt precursor or Fe(III) salt precursor.

10. The composition of claim 6, wherein the palladium is naturally present in the iron salt in amounts less than or equal to 1 ppm, 10 ppm, 50 ppm, 100 ppm, 200 ppm, 300 ppm, 400 ppm or 500 ppm relative to the iron salt or iron complex.

11. The composition of claim 10, where the amount of Pd present is controlled by external addition of a Pd salt to an iron salt.

12.-18. (canceled)

19. The composition of claim 7, wherein the iron is selected from the group consisting of a Fe(II) or Fe(III) salt, a Fe(II) salt precursor or Fe(III) salt precursor.

20. The composition of claim 7, wherein the palladium is naturally present in the iron salt in amounts less than or equal to 1 ppm, 10 ppm, 50 ppm, 100 ppm, 200 ppm, 300 ppm, 400 ppm or 500 ppm relative to the iron salt or iron complex.
